(19) United States
(12) Patent Application Publication
(10) Pub. No.: US 2011/0263015 A1
(43) Pub. Date: Oct. 27, 2011

(54) COMPOSITIONS AND METHODS FOR GENERATION OF PLURIPOTENT STEM CELLS

(75) Inventors: Jenice G. D'Costa, Gaithersburg, MD (US); Laurent M. Humeau, Germantown, MD (US); Stephen Gary Mansfield, Montgomery Village, MD (US); Madaiah Puttaraju, Germantown, MD (US); Nikolay Korokhov, Germantown, MD (US); Gerard J. McGarrity, Gaithersburg, MD (US)

(73) Assignee: VIRXSYS CORPORATION, GAITHERBURG (MD)

(21) Appl. No.: 13/059,951

(22) PCT Filed: Aug. 20, 2009
(86) PCT No.: PCT/US09/54375

§ 371 Date: Jun. 9, 2011

(30) Foreign Application Priority Data
Aug. 20, 2008 (US) 61/090348

(51) Int. Cl. C12N 5/071

(52) U.S. Cl. 435/366

(57) ABSTRACT

The present invention describes the use of pre-trans-splicing molecules (PTMs) to reprogram human normal and diseased somatic cells into pluripotent stem cells using spliceosome-mediated RNA trans-splicing. More specifically, the present invention describes the use of the SMaRT™ technology to repair or reprogram the newly induced diseased pluripotent stem cells.

### CROSS REFERENCE TO RELATED APPLICATIONS

This application claims priority to U.S. Provisional Application No. 61/090,348, filed Aug. 20, 2008, the contents of which are incorporated herein by reference in their entirety.

### FIELD OF THE INVENTION

This application relates to methods and compositions for the induction of pluripotent stem cells by spliceosome mediated RNA trans-splicing, and, more particularly, to methods and compositions comprising pre-trans-splicing molecules (PTMs) to generate pluripotent stem cells by reprogramming non-pluripotent cells via spliceosome mediated RNA trans-splicing (SMaRT™).

### BACKGROUND OF THE INVENTION

The derivation of human Embryonic Stem Cells (hESC) in 1998 generated much hope for the development of cell based therapies for degenerative and genetic diseases (Thomson, J. A., et al, Science 282, 1145-1147). However, the ensuing political, religious, social, and ethical controversies surrounding their derivation from human embryos has severely limited their therapeutic development for the clinic. Additionally, the consideration of graft rejection of the donor cells due to dissimilar immunological backgrounds is also a limiting factor in the clinical use of hESC.

The seminal work of Takahashi et al. and Yu et al. describing the derivation of induced Pluripotent Stem Cells (iPSC) from human skin fibroblasts in 2007, heralded a breakthrough not only for stem cell science, but also for their application as cell-based therapies for degenerative and genetic diseases (Takahashi et al., Cell 131, 861-72, 2007; Yu et al., Science 318, 1917-20, 2007).

The reprogramming of somatic cells iPSC has been described using retroviral and lentiviral vector delivery of cDNAs of 4 transcription factors, OCT3/4, SOX2, KLF4 and c-MYC (Takahashi et al., Cell 131, 1-12, 2007, herein incorporated by reference in its entirety), or OCT3/4, SOX2, NANOG and LIN28 (Yu et al., Science 1-4, 2007, herein incorporated by reference in its entirety), or OCT3/4, SOX2, KLF4, UTF1 or OCT3/4, SOX2, KLF4, c-MYC and p53 siRNA (Zhao et al., Cell Stem Cell 3, 475-479, herein incorporated by reference in its entirety).

The ability to generate patient-specific pluripotent stem cells without the technical intricacies and the ethical concerns surrounding somatic cell nuclear transfer opens the door for the treatment of limited conditions such as, for example, Parkinson's disease, cardiac failure, β islet replacement for type 1 diabetes, and correction of monogenic genetic diseases such as sickle cell anemia or hemophilia diseases, among others. Due to their therapeutic potential, the safety of generating iPSC for the clinic is a crucial aspect of this product development.

The two major safety issues facing iPSC generation/creation are the reactivation of oncogenic pluripotency factors used in their derivation, and the potential for insertional mutagenesis caused by integrating vectors used for delivery of the pluripotency factors.

SMaRT™ technology uses RNA molecules known as pre-trans-splicing molecules (PTMs) that are not translatable into proteins by themselves. A fully translatable transcript will be produced only when the endogenous targeted pre-mRNA and PTM are co-expressed in the same nucleus at the same time and undergo trans-splicing via the help of the modified cell spliceosome machinery. Thus, only the resulting mRNA transcript, fusion of the targeted endogenous pre-mRNA and the PTM, will encode for the protein of interest.

As stated, SMaRT™ technology is capable of reprogramming endogenous pre-mRNA using PTMs coding for pluripotency factor genes of interest. Further, trans-splicing of the pluripotency factors into endogenous pre-mRNA molecule selected targets that are progressively down-regulated or silenced as the cells are reprogrammed into iPSC, will ensure concomitant shut down of the pluripotency factors production once the cells are reprogrammed indicating the expression of these trans-spliced pluripotency factors are no longer required. This should also prevent inappropriate reactivation of oncogenic factor such as c-MYC, while the use of integration defective lentiviral vectors (LV) or other non integrating viral or non-viral delivery methods will circumvent insertional mutagenesis and concerns regarding transformation.

The use of integrating vectors generates a heightened level of safety concern compared to non-integrating delivery systems. In the case of the widely reported X-linked SCID (X-SCID) trials using a Murine leukemia (MLV) retroviral vector it is now thought that insertion of the vector activated an oncogene leading to leukemia in five out of twenty patients. Subsequent studies strongly suggest that the type of transgene and vector, the genetic disorder involved, and the selective advantage gained by cells that were corrected all combined for an unfavorable outcome. There is some evidence that integration of HIV-1-based vectors in hematopoietic cells can perturb the expression of some genes close to the integration site (600 kb), but global gene expression is unaltered. Other mouse models have been developed to assess the safety of LV and a majority of these studies have shown no association of LV transduction and integration with the development of either replication competent virus (RCL) or increased tumorigenesis.

Although lentiviral vectors integrate into the host genome, they can be produced as integration defective vectors by disrupting the integrase function of the HIV pol gene. This system will be transient in nature and will be progressively lost as the cells divide thus providing an additional safety layer. Due to the transient nature of the vector delivery system, the risk of reactivation of oncogenic pluripotency factors such as c-MYC or KLF4 will be further reduced. Additionally, integration defective vectors will also present much lower risk of insertional mutagenesis and activation or disruption of endogenous genes.

Accordingly, a continuing and unmet need exists for new, improved, safer, and alternative means for effective induction of pluripotent stem cells for research and therapeutic uses. This invention addresses these and other needs by the use of Splicesome-mediated RNA trans-splicing (SMaRT™) in conjunction with integration defective LV for the generation of iPSC.

Citation of the above documents or any references cited herein is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE INVENTION

The present invention provides induced pluripotent stem cells that are derived by reprogramming a non-pluripotent cell to a pluripotent stem cell using SMaRT™ technology. The present invention also provides methods for creation of iPSC that are derived by reprogramming a non-pluripotent cell to a pluripotent stem cell using RNA trans-splicing. The reprogramming step involves the introduction of at least one pre-trans-splicing RNA molecule (hereinafter, PTM) encoding a non-functional pluripotency factor into a non-pluripotent cell, which upon trans-splicing, produces a functional pluripotency factor that causes induction of the non-pluripotent cell to a pluripotent stem cell. The reprogramming step may also be carried out with non-pluripotent cells using at least one PTM encoding a non-functional pluripotency factor, in combination with a therapeutic product(s), which upon trans-splicing, produces a functional pluripotency factor that causes induction of the non-pluripotent cell to a pluripotent stem cell in which the therapeutic product(s) is expressed.

Thus, in one aspect of the invention, a non-pluripotent cell is provided comprising at least one PTM, which upon trans-splicing, produces a functional pluripotency reprogramming factor that causes induction of the non-pluripotent cell to a pluripotent stem cell.

In one embodiment of the present invention, the non-pluripotent cell is a differentiated adult or neo-natal somatic cell.

In one embodiment of the present invention, the pluripotency reprogramming factor can be a transcription factor.

Thus, in one embodiment of the present invention, the transcription factor can comprise a transcription factor encoded by an OCT family gene, a KLF family gene, a MYC family gene, and a SOX family gene, or any combination thereof. In yet another embodiment of the present invention, the transcription factor can comprise one or more gene products of each of: an OCT family gene, a KLF family gene, and a SOX family gene. In yet another embodiment of the present invention, the reprogramming transcription factor can further comprise one or more gene products of a SALL4 gene.

In yet another embodiment of the present invention, the transcription factor can comprise a gene product of the TERT gene in addition to a gene product of each of an OCT family gene, a KLF family gene, a MYC family gene, and a SOX family gene. In yet another embodiment of the present invention, one or more genes selected from the group consisting of the following genes: SV40 Large T antigen, HPV16 E6, HPV16 E7, and Bmil can be used in addition to a transcription factor encoded by an OCT family gene, the KLF family gene, the MYC family gene, the SOX family gene, and the TERT gene.

In yet another embodiment of the present invention, the transcription factor can comprise at least one of OCT3/4, SOX2, KLF4, c-MYC, NANOG, LIN 28, UTF1, p53 siRNA or any combination thereof.

In addition to these aforementioned transcription factors, the non-functional pluripotency factor encoded by the at least one PTM can further comprise a gene product or gene products of one or more kinds of genes selected from the group consisting of the following: FBX15, NANOG, ERAS, ECAT15-2, TCL1, and beta-catenin, ECAT1, ESG1, DNMT3L, ECAT8, Gdf3, SOX15, ECAT15-1, FTH117, SALL4, REX1, UTF1, STELLA, STAT3, and GRB2, or any combination thereof.

In yet another embodiment of the present invention, the transcription factors can include any of the aforementioned transcription factor(s), either with or without a transcription factor encoded by the MYC family gene.

In yet another embodiment of the present invention, each of the aforementioned pluripotency factor(s) may be used alone or in combination with other induced programmable stem cell factors as disclosed herein. In particular, each of the aforementioned pluripotency factor(s) may be used alone or in combination with other small molecules, compounds, or other agents relating to differentiation, development, proliferation or the like and factors having other physiological activities, as well as other gene products which can function as inducers of pluripotent stem cells such that reprogramming of non-pluripotent cell to induced pluripotent cells are obtained including, for example and not by way of limitation, histone deacetylase inhibitor such as valproic acid, histone methyltransferase inhibitor such as BIX-01294, Ca²⁺ channel activator Bay K 8644, components of the Wnt signaling pathway such as Wnt and β-catenin, or any combination thereof.

In another embodiment, the present invention also encompasses the generation of somatic cells or non-pluripotent fully differentiated cells derived by inducing differentiation of the aforementioned iPSC. The present invention thus provides a somatic cell or non-pluripotent fully differentiated cell derived by inducing differentiation of the aforementioned iPSC.

For example, and not by way of limitation, in yet another embodiment of the present invention, the transcription factor(s) can comprise one or more transcription factor(s) encoded by each of: an OCT family gene, a KLF family gene, in combination with a cytokine, chemokine or growth factor as described infra.

In yet another aspect of the present invention, a non-pluripotent cell is provided comprising at least one first PTM described above, and further comprising at least one second PTM encoding a therapeutic product, whereupon trans-splicing of the first PTM using SMaRT™ produces a functional pluripotency factor that causes induction of the non-pluripotent cell to a pluripotent stem cell; and whereupon trans-splicing of the second PTM using SMaRT™ results in expression of said therapeutic product.

In another embodiment of the present invention, the at least one first PTM and the at least one second PTM encoding a therapeutic product(s) are only functional after trans-splicing are co-expressed from the same vector, which upon trans-splicing via SMaRT™, cause induction of the non-pluripotent cell to a pluripotent stem cell in which the therapeutic product is expressed.

In yet another embodiment of the present invention, the at least one first PTM and the at least one second PTM encoding a therapeutic product(s) are expressed from separate vectors delivered either separately in any order or at the same time, which upon trans-splicing using spliceosome-mediated RNA trans-splicing, cause induction of the non-pluripotent cell to a pluripotent stem cell in which the therapeutic product is expressed.

In one embodiment of the present invention, a non-pluripotent cell is provided comprising at least one first PTM, and further comprising at least one second PTM encoding a therapeutic product, whereupon trans-splicing of the first PTM using SMaRT™ produces a functional pluripotency factor that causes induction of the non-pluripotent cell to a pluripotent stem cell, and whereupon trans-splicing of the second PTM using SMaRT™ results in expression of said therapeutic product so as to achieve a non-diseased or repaired pluripotent stem cell.

In yet another embodiment, the present invention also encompasses the generation of somatic cells or non-pluripotent fully differentiated cells derived by inducing differentiation of the aforementioned iPSC expressing the therapeutic product. The present invention thus provides a somatic cell or non-pluripotent fully differentiated cell derived by inducing differentiation of the aforementioned induced pluripotent stem cell in which the somatic cell or non-pluripotent fully differentiated cell expresses the therapeutic product.

In yet another embodiment, the present invention also encompasses the generation of somatic cells or non-pluripotent fully differentiated cells derived by inducing differentiation of the aforementioned induced pluripotent stem cells and then subsequently expressing a therapeutic product via trans-splicing.

Furthermore, in yet another embodiment of the present invention, the pluripotency factors encoded by the at least one PTM can comprise one or more microRNAs (“miRNAs”) or small interfering RNAs (siRNAs), which upon expression by trans-splicing, cause induction of the non-pluripotent cell to a pluripotent stem cell.

In one embodiment of the present invention, at least one PTM comprising one or more microRNAs (“miRNAs”) or small interfering RNAs (siRNAs) can be co-expressed from the same vector, which upon trans-splicing using SMaRT™, cause induction of the non-pluripotent cell to a pluripotent stem cell.

In yet another embodiment of the present invention, the at least one PTM comprising one or more microRNAs (“miRNAs”) or small interfering RNAs (SiRNAs) can be expressed from a separate vector delivered in any order, which upon trans-splicing using SMaRT™, cause induction of the non-pluripotent cell to a pluripotent stem cell. In one embodiment of the present invention, the miRNAs comprise miR-106a, miR-148a, miR-17, miR-182, miR-183, miR-183, miR-18^{a}, miR-18b, miR-19^{a}, miR-19b, miR-200c, miR-205, miR-20a, miR-20b, miR-25, miR302 cluster, miR-363, miR-92a, miR-92b, or any combination thereof.

In each of the aforementioned embodiments, the trans-splicing is mediated by SMaRT™. In another embodiment, the trans-splicing is mediated by Group I ribozymes. In yet another embodiment, the trans-splicing is mediated by Group II ribozymes.

In another aspect of the invention, a method is provided for generation of induced pluripotent stem cells comprising introducing into a non-pluripotent cell at least one PTM, which upon trans-splicing, produces a functional pluripotency factor that causes induction of the non-pluripotent cell to a pluripotent stem cell.

In one embodiment of the method of the present invention, the pluripotency factor(s) encoded by the at least one PTM can be a transcription factor comprising any one or more of the aforementioned transcription factors or a combination thereof.

In yet another embodiment of the method of the present invention, the transcription factor can comprise by way of example, and not by way of limitation, at least one of OCT3/4, SOX2, KLF4, c-MYC, NANOG, LIN 28, UTF1, p53 siRNA or any combination thereof.

In yet another embodiment, the present invention also provides such a method wherein the transcription factor can comprise one or more of the aforementioned transcription factor in combination with one or more of the cytokines, chemokines, or growth factors described infra.

The present invention also provides an induced pluripotent stem cell obtained by any of the aforementioned methods.

In another aspect of the invention, a method is provided for creation of induced pluripotent stem cells comprising introducing into a non-pluripotent cell at least one first PTM, and at least one second PTM encoding a therapeutic product(s), whereupon trans-splicing of the first PTM produces a functional pluripotency factor that causes induction of the non-pluripotent cell to a pluripotent stem cell, and whereupon trans-splicing of the second PTM results in the pluripotent stem cell expressing the therapeutic product(s).

In yet another embodiment of the method of the present invention, a non-pluripotent cell may be reprogrammed to an induced pluripotent stem cell using one or more of the aforementioned pluripotency factor(s). Thus, in one embodiment, therapeutic genes using PTMs can be delivered using SMaRT™ to the reprogrammed pluripotent stem cells such that spliceosome-mediated RNA trans-splicing will re-program defective transcripts only when the specific targeted endogenous pre-mRNA is expressed in progeny cells. Thus, in one embodiment of the present invention, a method is provided for creation of healthy iPSC comprising introducing into a diseased non-pluripotent cell at least one first PTM as described above, and at least one second PTM encoding a therapeutic product(s), whereupon trans-splicing of the first PTM produces a functional pluripotency factor that causes induction of the non-pluripotent cell to a pluripotent stem cell, and whereupon trans-splicing of the second PTM results in the generation of a pluripotent stem cell expressing the therapeutic product(s).

In yet another aspect, the present invention also provides for the generation of somatic cells derived by inducing differentiation of the aforementioned induced pluripotent stem cells. The present invention thus provides a somatic cell derived by inducing differentiation of the aforementioned induced pluripotent stem cells.

Thus, in one embodiment, the present invention comprises a method for stem cell therapy in a patient comprising: isolating and collecting a non-pluripotent cell from a patient; inducing said pluripotent cell from the patient into an induced pluripotent stem cell by introducing at least one PTM, which upon trans-splicing, produces a functional pluripotency factor that causes induction of the non-pluripotent cell to a pluripotent stem cell; inducing differentiation of said induced pluripotent stem cell, and transplanting the differentiated cell into said patient.

In each of the aforementioned embodiments of the methods of the present invention, the trans-splicing is mediated by SMaRT™. In another embodiment, the trans-splicing is mediated by Group I ribozymes. In yet another embodiment, the trans-splicing is mediated by Group II ribozymes.

These and other aspects of some exemplary embodiments will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments without departing from the spirit thereof. Additional features may be understood by referring to the accompanying drawings, which should be read in conjunction with the following detailed description and examples.

### BRIEF DESCRIPTION OF THE FIGURES

FIG. 1 Schematic representation of reprogramming and gene correction or therapeutic protein expression using PTM's targeted to differentiation stage specific endogenous pre-mRNA's in non-pluripotent cells and their re-programmed and differentiated products [NPC: non-pluripotent cell; DC: differentiated cell].

FIG. 2 schematically illustrates the use of spliceosome mediated RNA trans-splicing (SMaRT™) to express proteins from endogenous pre-mRNA targets that are preferentially expressed in the starting non-pluripotent cell populations, but that are progressively downregulated or shut off as the cells are reprogrammed into induced pluripotent stem cells with concomitant loss of transcription factor expression.

FIG. 3 schematically illustrates spliceosome mediated RNA trans-splicing of pluripotency transcription factor OCT3/4 to endogenous target Ribosomal protein L10 and expression of chimeric OCT3/4 protein.

FIG. 4 schematically illustrates the use of SMaRT™ technology to reprogram somatic cells to pluripotent stem cells and for the therapeutic correction of diseased pluripotent stem cells with a second PTM.

FIG. 5 schematically illustrates some of the potential lentiviral vector configurations for the delivery of PTMs into cells.

FIG. 6 schematically illustrates the therapeutic use of SMaRT™ technology in treatment of Cystic Fibrosis via gene therapy involving stem cells, by using PTM for repair of the defective CFTR pre-mRNA.

FIG. 7 schematically illustrates the therapeutic use of SMaRT™ technology in the repair of Factor VIII pre-mRNA in hepatocytes for the treatment of Hemophilia-A. The human Factor VIII (FVIII) PTM binds to intron 22 of the mutant human FVIII.

FIG. 8 schematically illustrates a human FVIII binding to binding to the von Willebrand Factor pre-mRNA in Liver Sinusoidal Endothelial Cells (LSEC) and the process by which functional FVIII is generated.

FIG. 9 schematically illustrates trans-splicing of a human FVIII PTM to human albumin pre-mRNA (reprogramming) to create high levels of circulating FVIII, in order to treat Hemophilia-A patients.

FIG. 10 schematically illustrates the treatment of dementia via reprogramming of mutant microtubule associated protein tau splicing, with the help of SMaRT™ technology using a PTM containing human tau exon.

FIG. 11 schematically illustrates the repair of human serpin A1 with SMaRT™ technology, in treating protein alpha1 anti-trypsin (AAT) deficiency relating to lung and liver disease.

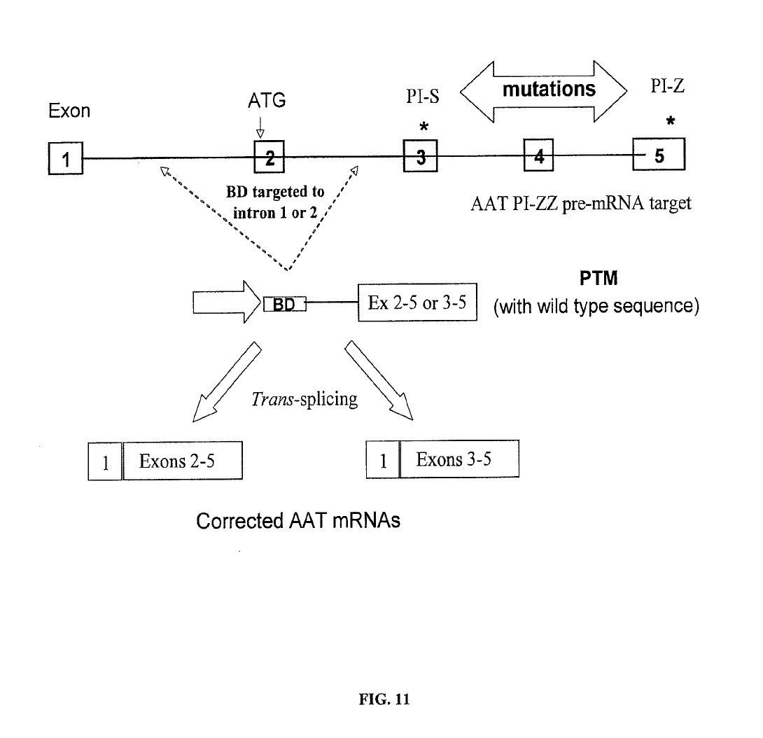

The maps illustrated in the drawings are not drawn to scale, and the relative sizes of particular segments or functional elements are not necessarily proportional to the lengths (e.g., number of base pairs) of the corresponding sequences.

### DETAILED DESCRIPTION OF THE INVENTION

Provided herein are PTMs to reprogram human normal and diseased somatic cells to pluripotent stem cells using spliceosome-mediated RNA trans-splicing. The reprogramming of somatic cells into pluripotent stem cells involves the introduction in said somatic cells of PTMs that encode for not fully and directly translatable RNA transcripts for pluripotency reprogramming factors, but upon trans-splicing with pre-mRNA target molecules in the nucleus of said somatic cells, create trans-spliced mRNA molecules that are now fully translatable into proteins which cause induction of said somatic cell to a pluripotent stem cell and/or provide a therapeutic benefit.

### DEFINITIONS

As used herein, each of the following terms has the meaning associated with it in this section.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the present invention, the following terms are defined below.

As used in this specification and the appended claims, the singular forms “a,” “an,” and “the” include plural referents unless the content clearly dictates otherwise. Thus, for example, reference to “a vector” may include a combination of two or more vectors, reference to “DNA” may include mixtures of DNA, and the like.

The term “non-pluripotent cells” refers to those cells that have limited self-renewal capacities and are already committed to a tissue, and include for example, and not by way of limitation, somatic cells and lineage committed progenitor cells.

The term “induced pluripotent stem cells (iPS cells or iPSC)” refers to those non-pluripotent cells that have been induced to dedifferentiate so as to be pluripotent, as defined by unlimited capacities for self renewal and to differentiate into all cell lineages.

The term “induction” refers to the process whereby a non-pluripotent cell is reprogrammed to become an induced pluripotent stem cell.

A “ribozyme sequence” is a catalytic RNA sequence capable of cleaving a target RNA, such as a hairpin or hammerhead ribozyme. The term also encompasses a nucleic acid sequence in an expression cassette from which the RNA is transcribed.

A “miRNA” or microRNA is a single-stranded RNA molecule of about 21 to 23 nucleotides in length, which regulate gene expression. miRNAs are encoded by genes that are transcribed from DNA, but not translated into protein. They are processed from primary transcripts known as pri-miRNA to short stem-loop structures called pre-miRNA and finally to functional miRNA. Their main function is to downregulate gene expression.

The term “subsequence” in the context of a particular nucleic acid sequence refers to a region of the nucleic acid equal to or smaller than the specified nucleic acid.

Pre-Trans-Splicing Molecules (PTMs) Encoding Pluripotency Inducing Factors

The present invention relates to induced pluripotent stem cells that are derived by reprogramming a non-pluripotent cell to a pluripotent stem cell using RNA trans-splicing via SMaRT™.

In one aspect of the invention, a non-pluripotent cell is provided comprising at least one PTM, which upon trans-splicing, produces a functional pluripotency factor that causes induction of the non-pluripotent cell to a pluripotent stem cell. In one embodiment of the present invention, the pluripotency factor encoded by the at least one PTM can be a transcription factor. In one embodiment of the present invention, the transcription factor encoded by the at least one PTM can comprise a transcription factor encoded by an OCT family gene, a KLF family gene, a MYC family gene, and a SOX family gene, or a combination thereof. In yet another embodiment of the present invention, the transcription factor encoded by the at least one PTM can comprise one or more gene products of each of: an OCT family gene, a KLF family gene, and a SOX family gene. In yet another embodiment of the present invention, the transcription factor encoded by the at least one PTM can comprise one or more gene products encoded by each of: an OCT family gene, a KLF family gene, a MYC family gene, and a SOX family gene. In yet another embodiment of the present invention, the transcription factor encoded by the at least one PTM can further comprise one or more gene products of a SALL4 gene.

With regard to gene family members, the at least one PTM encoding a transcription factor may comprise any combination of members from one or more gene families. For example, a combination of at least one PTM encoding one or more gene products of OCT3/4, KLF4, and c-MYC may be used as the transcription factor. Examples of the OCT family transcription factor encoded by the at least one PTM can include, for example, OCT3/4, OCT1A, OCT6, and the like. OCT3/4 is a transcription factor belonging to the POU family, and is reported as a marker of undifferentiated cells (Okamoto et al., Cell 60:461-72, 1990, herein incorporated by reference in its entirety). OCT3/4 is also reported to participate in the maintenance of pluripotency (Nichols et al., Cell 95:379-91, 1998, herein incorporated by reference in its entirety). Examples of the KLF family transcription factor encoded by the at least one PTM can include Klf1, Klf2, Klf4, Klf5 and the like. Klf4 (Kruppel like factor-4) is reported as a tumor repressing factor (Ghaleb et al., Cell Res. 15:92-96, 2005, herein incorporated by reference in its entirety). Examples of the MYC family transcription factor encoded by the at least one PTM can include c-MYC, N-MYC, L-MYC and the like. c-MYC is a transcription control factor involved in differentiation and proliferation of cells (Adhikary & Eilers, Nat. Rev. Mol. Cell. Biol. 6:635-45, 2005, herein incorporated by reference in its entirety), and is also reported to be involved in the maintenance of pluripotency (Cartwright et al., Development 132:885-96, 2005, herein incorporated by reference in its entirety).

In each of the embodiments of the compositions and methods of the present invention recited herein, the nucleotide sequences encoding the non-functional pluripotency factors or variants thereof may be obtained directly from the nucleotide sequences of such pluripotency factors or from genes encoding such pluripotency factors that are currently available or become available in public databases such as, for example, and not by way of limitation, those set forth in NCBI, Gen Bank by using the provided accession numbers set forth herein as if such pluripotency factor nucleotide sequences were set forth specifically herein in their entirety.

The NCBI accession numbers of the genes of the families other than Oct3/4, Klf4 and c-MYC are as follows (in each instance listed as mouse and human, respectively, and in each instance, each nucleotide sequence depicted in the text of the accession number entry is specifically herein incorporated by reference in its entirety): Klf1 Kruppel-like factor 1 (erythroid) [NM_{—}010635/NM_{—}006563]; Klf2 Kruppel-like factor 2 (lung) [NM_{—}008452 NM_{—}016270]; Klf5 Kruppel-like factor 5 [NM_{—}009769/NM_{—}001730]; c-MYC myelocytomatosis oncogene [NM_{—}010849/NM_{—}002467]; N-MYC v-MYC myelocytomatosis viral related oncogene [NM_{—}008709/NM_{—}005378]; L-MYC v-MYC myelocytomatosis viral oncogene homolog 1, lung carcinoma derived (avian) [NM_{—}008506/NM_{—}005376]; Oct1A POU domain, class 2, transcription factor 1 [NM_{—}198934/NM_{—}002697]; Oct6 POU domain, class 3, transcription factor 1 [NM_{—}011141/NM_{—}002699].

The pluripotency reprogramming factor of the present invention may comprise a transcription factor other than the aforementioned three kinds of transcription factor (i.e., OCT3/4, KLF4, and c-MYC). An example of such transcription factor includes a gene product of a SOX family gene. Examples of the SOX family transcription factors encoded by the at least one PTM can include, for example, SOX1, SOX3, SOX7, SOX15, SOX17 and SOX18, and a preferred example includes SOX2. A transcription factor comprising at least a combination of the gene products of four kinds of genes, an OCT family gene (for example, OCT3/4), a KLF family gene (for example, KLF4), a MYC family gene (for example, c-MYC), and a SOX family gene (for example, SOX2), each either encoded by the at least one PTM (or not) is one non-limiting example of a preferred embodiment of the present invention with respect to reprogramming efficiency, and in particular, a combination of a transcription factor of a SOX family gene encoded by the at least one PTM is sometimes preferred to obtain induced pluripotent stem cells exhibiting pluripotency. SOX2, expressed in an early development process, is a gene encoding a transcription factor (Avilion et al., Genes Dev. 17:126-40, 2003, herein incorporated by reference in its entirety). The NCBI accession numbers of SOX family genes other than SOX2 are as follows (in each instance listed as mouse and human, respectively, and in each instance, each nucleotide sequence depicted in the text of the accession number entry is specifically herein incorporated by reference in its entirety): SOX1 SRY-box containing gene 1 [NM_{—}009233/NM_{—}005986]; SOX3 SRY-box containing gene 3 [NM_{—}009237/NM_{—}005634]; SOX7 SRY-box containing gene 7 [NM_{—}011446/NM_{—}031439]; SOX15 SRY-box containing gene 15 [NM_{—}009235/NM_{—}006942]; Sox17 SRY-box containing gene 17 [NM_{—}011441/NM_{—}022454]; SOX18 SRY-box containing gene 18 [NM_{—}009236/NM_{—}018419]

In yet another embodiment of the present invention, a transcription factor of a MYC family gene encoded by the at least one PTM may be replaced with a cytokine, chemokine, or growth factor. As the cytokine, for example, SCF, bFGF or the like is preferred. However, other cytokines, chemokines, or growth factors may be used including, for example and not by way of limitation, neural growth factors, hematopietic growth factors, interleukins, hepatocyte growth factors, growth factors for pancreatic cells, other growth factors/modulators described infra, or any combination thereof.

In yet another preferred embodiment, an example of a transcription factor(s) includes a factor encoded by the at least one PTM which induces immortalization of cells, in addition to the aforementioned three kinds of transcription factors (i.e., OCT3/4, KLF4, and c-MYC), preferably, the four kinds of transcription factors (i.e., OCT3/4, Klf4, c-MYC, and SOX). For example, an example includes a combination of a transcription factor(s) comprising a gene product of the TERT gene encoded by the at least one PTM. In yet another embodiment, the transcription factor(s) comprises any of the aforementioned gene products in combination with a pluripotency factor comprising a gene product or gene products of one or more kinds of the following genes: SV40 Large T antigen, HPV16 E6, HPV16 E7, and Bmil encoded by the at least one PTM. In particular, TERT is essential for the maintenance of the telomere structure at the end of chromosome at the time of DNA replication, and the gene is expressed in stem cells or tumor cells in humans, whilst it is not expressed in many somatic cells (Horikawa et al., P.N.A S. USA 102:18437-442, 2005). SV40 Large T antigen, HPV16 E6, HPV16 E7, or Bmil was reported to induce immortalization of human somatic cells in combination with Large T antigen (Akimov et al., Stem Cells 23:1423-33, 2005; Salmon et al., Mol. Ther. 2:404-14, 2000). These aforementioned pluripotency factors are extremely useful particularly when induced pluripotent stem cells or cell lines are being induced from human non-pluripotent cells. The NCBI accession numbers of TERT and Bmil genes are as follows (in each instance listed as mouse and human, respectively, and in each instance, each nucleotide sequence depicted in the text of the accession number entry is specifically herein incorporated by reference in its entirety): TERT telomerase reverse transcriptase [NM_{—}009354/NM_{—}198253]; Bmil B lymphoma Mo-MLV [NM_{—}007552/NM_{—}005180] insertion region 1.

Furthermore, a pluripotency factor(s) of one or more kinds of genes selected from the group consisting of the following: Fbx15, Nanog, ERas, ECAT15-2, Tcl1, and .beta.-catenin (either alone or in combination) encoded by the at least one PTM may be combined. In yet another preferred embodiment, from a viewpoint of non-pluripotent cell reprogramming efficiency, an example includes pluripotency factor(s) comprising a total of ten kinds of gene products, wherein gene products of Fbx15, Nanog, ERas, ECAT15-2, Tcl1, and .beta.-catenin encoded by the at least one PTM are combined with the aforementioned four kinds of transcription factors. Fbx15 (Tokuzawa et al Mol. Cell. Biol. 23:2699-708, 2003, herein incorporated by reference in its entirety), Nanog (Mitsui et al., Cell 113:631-42, 2003, herein incorporated by reference in its entirety), ERas (Takahashi et al. Nature 423:541-45, 2003, herein incorporated by reference in its entirety), and ECAT15-2 ((Imamura et al. BMC Dev Biol. 2006; 6: 34); Bortvin et al., Development 130:1673-80, 2003, herein incorporated by reference in their entirety) are genes specifically expressed in embryonic stem cells. TCL1 is involved in early embryonic development in mice (Narducci et. al. PNAS 99; 11712-11717, 2002, herein incorporated by reference in its entirety), and beta-catenin is an important factor constituting the Wnt signal transmission pathway, and also reported to be involved in the maintenance of pluripotency (Sato et al, Nat. Med. 10:55-63, 2004, herein incorporated by reference in its entirety).

In yet another embodiment, the pluripotency factor(s) of the present invention may comprise, for example, a pluripotency factor(s) of one or more kinds of genes selected from the group consisting of the following: ECAT1, Esg1, Dnmt3L, ECAT8, Gdf3, SOX15, ECAT15-1, Fthl17, SALL4, Rex1, UTF1, Stellar, Stat3, and Grb2 (either alone or in combination) encoded by the at least one PTM. ECAT1, Esg1, ECAT8, Gdf3, and ECAT15-1 are genes specifically expressed in ES cells (Mitsui et al., Cell 113:631-42, 2003). Dnmt3L is a DNA methylating enzyme-related factor, and SOX 15 is a class of genes expressed in an early development process and encoding transcription factors (Maruyama et al., J. Biol. Chem. 280:24371-79, 2005, herein incorporated by reference in its entirety). FTHL17 encodes ferritin heavy polypeptide-like 17 (Loriot et al., Int. J. Cancer 105:371-76, 2003, herein incorporated by reference in its entirety; Eamon Geoghegan and Lucy Byrnes, Int. J. Dev. Biol. 52: 1015-1022, 2008, herein incorporated by reference in its entirety), SALL4 encodes a Zn finger protein abundantly expressed in embryonic stem cells (Kohlhase et al., Cytogenet. Genome Res. 98:274-77, 2002, herein incorporated by reference in its entirety), and Rex1 encodes a transcription factor locating downstream from OCT3/4 (Ben-Shushan et al., Mol. Cell. Biol. 18:1866-78, 1998, herein incorporated by reference in its entirety). UTF1 is a transcription cofactor locating downstream from OCT3/4, and it is reported that the suppression of the proliferation of ES cells is induced when this factor is suppressed (Okuda et al., EMBO J. 17:2019-32, 1998, herein incorporated by reference in its entirety). Stat3 is a signal factor for proliferation and differentiation of cells. The activation of Stat3 triggers the operation of LIF, and thereby the factor plays an important role for the maintenance of pluripotency (Niwa et al., Genes Dev. 12:2048-60, 1998, herein incorporated by reference in its entirety). Grb2 encodes a protein mediating between various growth factor receptors existing in cell membranes and the Ras/MAPK cascade (Cheng et al. Cell 95:793-803, 1998, herein incorporated by reference in its entirety).

In each embodiment of the compositions of the aforementioned pluripotency factor(s) of the present invention and methods of using same as described in detail herein, the pluripotency factor(s) encoded by the at least one PTM also specifically includes those derivatives, fragments or modifications thereof, which upon trans-splicing, produces a functional pluripotency factor that causes induction of the non-pluripotent cell to a pluripotent stem cell. Any and all such nucleotide variations and resulting amino acid polymorphisms or variations of the pluripotency factor(s) described herein that are the result of natural genotypic, allelic variation, or that have been artificially engineered, and which, upon trans-splicing, produces a functional pluripotency factor that causes induction of the non-pluripotent cell to a pluripotent stem cell, are intended to be within the scope of the invention.

Thus, derivatives, fragments or modifications thereof of the pluripotency factor(s) encoded by the at least one PTM can be created by introducing one or more nucleotide substitutions, additions or deletions into the nucleotide sequence of the pluripotency factor(s), such that one or more amino acid residue substitutions, additions, or deletions are introduced into the pluripotency factor(s) encoded by the at least one PTM. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence of the pluripotency factor(s), such as by saturation mutagenesis, and the resultant mutants can be screened for biological activity to identify mutants that, upon transplicing using SMaRT™, produce a functional pluripotency factor that causes induction of the non-pluripotent cell to a pluripotent stem cell.

In yet another embodiment of the present invention, any of the aforementioned pluripotency factor(s) encoded by the PTM can be used in combination with a cytokine, chemokine or growth factor as described supra. In one embodiment of the present invention, for example and not by way of limitation, the cytokine, chemokine or growth factor comprise neural growth factors, hematopietic growth factors, interleukins, hepatocyte growth factors, growth factors for pancreatic cells, other growth factors/modulators described infra, or any combination thereof.

In one embodiment, the neural growth factors comprise one or more of Amphiregulin, betacellulin, epidermal growth factor (EGF), EGF-L6, epigen, epiregulin, heparin-binding EGF, LRIG1 (leucine-rich repeat and Ig-like domain-containing-1), LRIG3, Neuregulin-1, neuregulin-3, TGF-α, TMEFF1, TMEFF2, ErbB2, EbrB3, ErbB4, herstatin, activin A, Activin AB, Activin AC, Activin B, Activin C, Fibroblast growth factor (FGF) acidic, FGF basic, FGF-3-6, FGF-8-13, FGF-16-17, FGF-19-23, FGF-7, FGF-BP, FRS2, Klotho, Klotho beta, LIN41, alpha 2-macroglobulin, pentraxin 3, SPRY3, growth/differentiation factor (GDF)-1, -3, -5, -6, -7, -8, -9, -11, -15, Desert heggehog, Indian hedgehog, Sonic hedgehog, GSK-3 alpha/beta, Hip, LIN-41, Patched, Patched 2, GLI-1, -2, -3, glypican 3, insulin growth factor (IGF)-I, IGF-II, acid labile subunit, CILP-1, CTGF, Cyr61, Endocan, IGFBP-1, -2, -3, -4, -5, -6, -L1, -rP10, IMP2, NOV/CCN3, WISP-1/CCN4, stem cell-derived neural stem/progenitor cell supporting factor, activin, activin RIA/ALK-2, Activin RIB/ALK-4, Activin RIIA, Activin RIIA/B, Activin RIIB, ALK-1, ALK-7, BMPR-IA/ALK-3, BMPR-IB/ALK-6, BMPR-II, CD109, cripto, endoglin/CD105, GFP alpha-1/GDNF R alpha-1, -2, -3, -4, MIS RII, NCAM-1/CD56, Ret, RGM-A, -B, -C, TGF-beta RI, -RII, -RIIb, -RIII, Neuropilin-1, -2, PDGF, PDGF R beta, PDGF-AB, -b, -c, -d, -A, -AA, -BB, -CC, -DD, PIGF, PIGF-2, VEGF, VEGF/PIGF heterodimer, VEGF-B, -C, -D, -R, VEGF R1/Flt-1, VEGF R2/KDR/Flk-1, VEGF R3/Flt4, Wnt-1, -2, -2b, -3a, -4, -5a, -5b, -6, -7a, -7b, -8a, -8b, -9a, -9b, -10a, -10b, -11, glypican 1, glypican 3, glypican 5, kremen-1, kremen-2, MESDC2, MFRP, myocilin, norrin, nucleoredoxin, R-spondin 1, R-spondin 2, 3, 4, Scleostin, BMP-2, BMP-2/BMP-7 heterodimer, BMP-2/BMP-4 cross reactive, BMP-3, BMP-3b, BMP-4, BMP-4/BMP-7 heterodimer, BMP-5, -6, -7, -8, -9, -10, -15, -2a, decapentaplegic, Glial-Derived Neurotrophic Factor, artemin, Brain Derived Neurotrophic Factor (BDNF), beta-NGF, Ciliary Neurotrophic Factor (CNTF), Glia Maturation Factor beta, midkine, neuritin, neurturin, Neurotrophin-3, Neurotrophin-4, persephin, pleiotrophin, Slit2-N, beta-nerve growth factor, neuropilin-1, neuropilin-2, or any combination thereof.

In one embodiment, the hematopietic growth factors comprise one or more of Stem cell factor, IL-3, IL-6, Thrombopoietin, Flt-3, M-CSF, GM-CSF, Erythropoietin, G-CSF, IL-7, Recombinant Human 4-1BB Ligand, Stem Cell Growth Factor-β, Stem Cell Growth Factor-α, or any combination thereof.

In one embodiment, interleukins comprise one or more of IL-1, IL-1beta, IL-1Ra, IL-2, sIL-2Rα, IL-3, IL-4, IL-4Rα, IL-5, IL-6, sIL-6Rα, IL-7, IL-8, IL-9, IL-10, IL-11, IL-12, IL-13, IL-14, IL-15, IL-16, IL-17, IL-17A-F, IL-18, IL-19, IL-20, IL-21, IL-22, IL-31, IL-33, or any combination thereof.

In one embodiment, the hepatocyte growth factors comprise one or more of HGF/Scatter factor/hepatopoietin, Macrophage-stimulating protein (MSP), Activin family, BMP family, Hedgehog family, FGF family, GDF family, Oncostatin, or any combination thereof.

In one embodiment, growth factors for pancreatic cells comprise one or more of activin, Wnt, KAAD-cyclopamine, human fibroblast growth factor 10 (FGF-10), retinoic acid, γ secretase inhibitor, extendin, insulin growth factor 1 (IGF1), hepatocyte growth factor 1 (HFG1), or any combination thereof.

In one embodiment, the other growth factors/modulators comprise one or more of 4-1BBL, Klotho, leptin, LIGHT, maspin, MIA/Melanoma Inhibitory Activity/Cartilage-derived retinoic acid-sensitive protein (CD-RAP), MIA2, noggin, Novel Neurtrophin-1/B-Cell Stimulating Factor-3/Cardiotrophin-like cytokine, Nephroblastoma Overexpressed gene (NOV), cardiotropin, myostatin, myostatin-propeptide, oncostatin M, Oncostatin-M (209aa), Osteoprotegrin, OTOR, OX40 ligand, Placenta Growth Factor-1/PIGF/PGF, PIGF-2, PIGF-3, Prokinecticin-2, prolactin, Parathyroid Hormone-related Protein, relaxin-2, relaxin-3, RELM-β, resistin, sCD14, sCD23, sCD40 ligand, sDelta-like protein 1, sDelta-like protein 4, Soluble Frizzled Related Protein-1, soluble Fas ligand, RANK ligand, sTNF receptor type I, -type II, soluble TRAIL receptor-1, -2, Transmembrane activator and CAML interactor, TAFA-2, transforming growth factor-α, transforming growth factor-β1, transforming growth factor-β2, β3, TL-1A, leptin, maspin, Toll-like receptor 3/CD283 antigen, TNF-α, TNF-β, TRAIL, Trefoil factor 1, trefoil factor 2, trefoil factor 3, Twisted Gastrulation Protein, Thymic Stromal Lymphopoietin, TNF-related weak inducer of apoptosis (TWEAK), TWEAK receptor, vaspin, vascular endothelial growth factor family (VEGF), VEGF-B, VEGF-C, VEGF-D, visfatin, WNT-1 inducible signaling pathway protein-1 (WISP), angiopoietin-1, A Proliferating-inducing Ligand (APRIL), Activation-induced TNFR member Ligand (AITRL), B cell Activating Factor belonging to the TNF family (BAFF), BAFF receptor, B-Cell Maturation Antigen (BCNA), betacellulin, cardiotrophin-1, CD22, Connective Tissue Growth Factor, Connective Tissue Growth Factor-Like protein (WISP-2), CYR61, DKK-1, DKK-4, EG-VEGF, HB-EGF, EMAP-II, endostatin, follistatin, adipolean, galectin-1, galectin-3, HVEM, Intercellular adhesion molecule 1, INF-α, INF-beta, INF-γ, INF-λ1, INF-λ2, Insulin-like Growth Factor-Binding Protein 1, Insulin-like Growth Factor-Binding Protein 2, Insulin-like Growth Factor-Binding Protein 3, Insulin-like Growth Factor-Binding Protein 4, Insulin-like Growth Factor-Binding Protein 5, Insulin-like Growth Factor-Binding Protein 6, Insulin-like Growth Factor-Binding Protein 7, Insulin-like Growth Factor-I, Insulin-like Growth Factor-2, matrix metalloproteinase family, TIMP family, Platelet derived growth factor family, VEGF family, Insulin-like growth factor family, Insulin-like growth factor binding protein family, or any combination thereof.

In yet another aspect of the present invention, the target or target binding domain for the pluripotency factor encoded by the at least one PTM can comprise RPL10, ESE3, basonuclin, RPL37, or RPL3, or any combination thereof.

In one embodiment, the target or target binding domain for the pluripotency factor encoded by the at least one PTM can comprise pre-mRNA transcripts that are preferentially expressed in non-pluripotent cells such as fibroblasts and keratinocytes. These pre-mRNA transcripts for fibroblasts cells include for example, and not by way of limitation, those pre-mRNA transcripts specifically listed in Supplemental Data S-Table 4: “Genes showing more than 5-fold increase in HDF than in human iPS cells.” (Cell, Volume 131, Issue 5, 861-872, 30 Nov. 2007, herein incorporated by reference in its entirety).

In another embodiment, the target or target binding domain for the pluripotency factor encoded by the at least one PTM can comprise those pre-mRNA transcripts that show preferential expression in keratinocytes compared to iPSC and that are disclosed in “Nat Biotechnol. 2008 November; 26 (11):1276-84. Epub OCT 17, 2008, Supplemental data”, the entire contents of which are incorporated herein by reference.

In yet another embodiment, the target or target binding domain for the pluripotency factor encoded by the at least one PTM can comprise those pre-mRNA transcripts that are highly expressed in CD34+ cells and are disclosed in Ivanova et al. Science 18 Oct. 2002: 601, Table S2. 2728 (Ivanova et al. Science 18 Oct. 2002: 601, Table S2. 2728 cDNAs enriched in Hematopoietic Stem Cell populations. Functional annotations based on PubMed, LocusLink, UniGene and OMIM. Motif detection based on Prosite profiles and Pfam hidden Markov models after EST assembly, the entire contents of which are incorporated herein by reference).

In yet another embodiment, the target or target binding domain for the pluripotency factor encoded by the at least one PTM can comprise those pre-mRNA transcripts of CD34+ cells which have similar expression patterns to iPSC which pre-mRNA transcripts have been reported in “PNAS 2009 106:8278-8283; Table 4, Table 5 and Supplementary Tables 5A, 5B, 5C, 5D and 5E,” the entire contents of which are incorporated herein by reference In another embodiment, a non-pluripotent cell of the invention can further comprise a first PTM, described herein, encoding at least one pluripotency inducing factor, which upon trans-splicing, produces a functional pluripotency factor that causes induction of the non-pluripotent cell to a pluripotent stem cell; and a second PTM encoding a therapeutic nucleic acid sequence for replacing at least a portion of a defective gene in the induced pluripotent cell with the therapeutic nucleic acid sequence.

The therapeutic nucleic acid sequence can encode, by way of example, not limitation, all or part of genes selected from the group consisting of encodes all or part of cystic fibrosis transmembrane conductance regulator protein (CFTR); Factor VIII; Apolipoprotein A-1; alpha1 anti-trypsin; globin; insulin; glucagon-like peptide-1, or any combination thereof, which upon trans-splicing using SMaRT™, corrects the defective gene in the induced pluripotent stem cell. In another embodiment, the therapeutic nucleic acid can encode all or a part of any gene that contributes to disease in the iPSC.

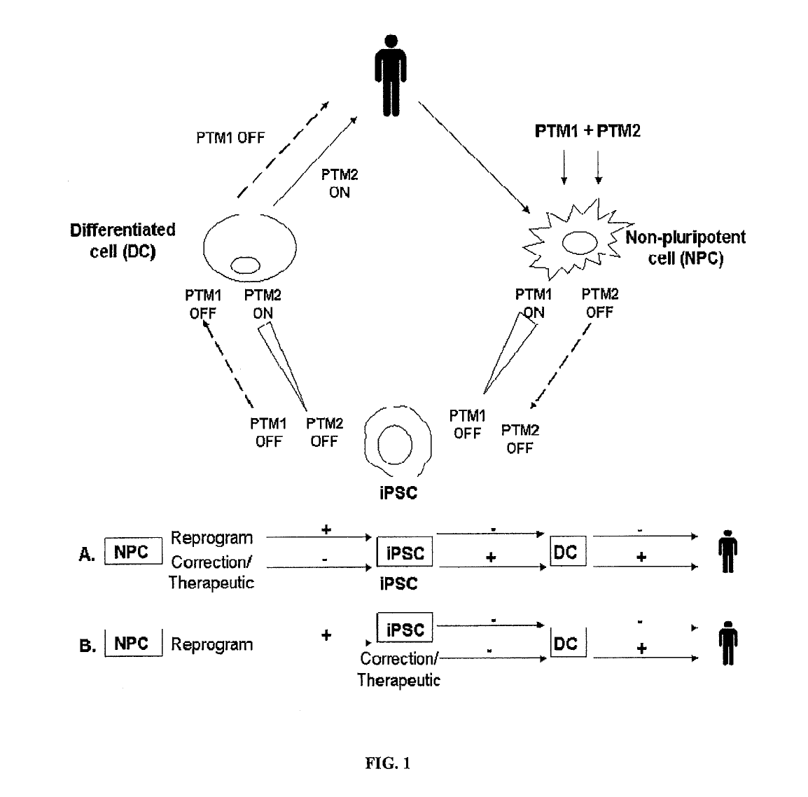

Now referring specifically to the attached drawings, in one embodiment of the present invention, a schematic representation of reprogramming and gene correction or therapeutic protein expression using PTM's targeted to differentiation stage specific endogenous pre-mRNA's in non-pluripotent cells and their re-programmed and differentiated products is depicted in FIG. 1 where NPC is non-pluripotent cell and DC is differentiated cell. In particular, referring specifically to FIG. 1, non-pluripotent cells (e.g. fibroblasts, keratinocytes, CD34+ cells) from the patient are harvested and cultured in appropriate medium. The reprogramming PTMs coding for pluripotency factors (PTM set 1) and PTM's to correct the gene defect or to express a therapeutic protein (PTM set 2) are introduced into the cells using, for example, and not by way of limitation, retroviral vectors, lentiviral vectors, adeno-associated viral based vectors, adenoviral vectors, viral vector transduction, electroporation, transformation, transduction, conjugation, transfection, infection, membrane fusion with cationic lipids, high-velocity bombardment with DNA-coated microprojectiles, incubation with calcium phosphate-DNA precipitate, or direct microinjection into single cells. The reprogramming PTM set 1 is targeted to endogenous pre-mRNAs that are expressed in the non-pluripotent cells, and following trans-splicing, becomes fully translatable and causes re-programming into iPSC. On re-programming, the endogenous target pre-mRNA is down-regulated or silenced, and thus PTM set 1 is not trans-spliced at all and no functional transcripts are produced once cells become iPS cells. PTM set 2 is targeted to endogenous pre-mRNAs that are not expressed either in the non-pluripotent cells or the iPS cells, and hence are not trans-spliced at all and no functional transcripts are produced at these stages in the process. iPSC can be differentiated to various cell lineages using defined culture conditions. PTM set 2 encoding either a therapeutic protein, is targeted to endogenous pre-mRNAs that are expressed in specific differentiation stages or cell lineages, and thus are trans-spliced into fully translatable transcripts only when the iPS cells differentiates into the specified cell lineage. The differentiated cell that is now either gene corrected or expressing the therapeutic protein is transplanted back into the patient (FIG. 1 A). Alternatively, PTM set 2 can be introduced into iPSC on completion of re-programming (FIG. 1 B)

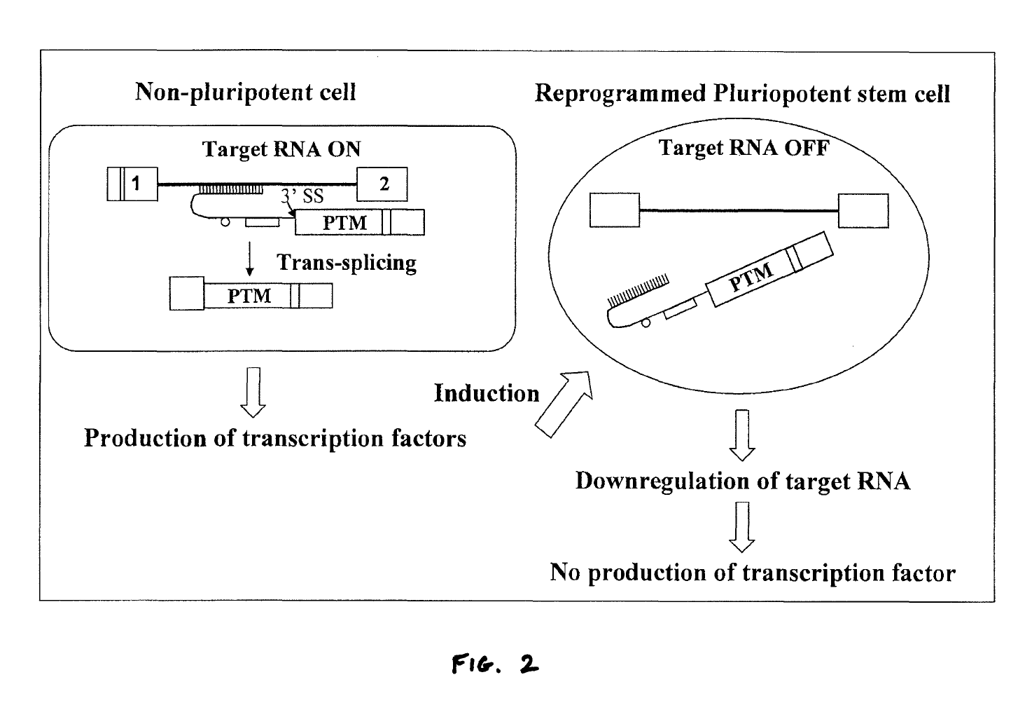

FIG. 2 describes a schematic representation of SMaRT™ technology (as described in U.S. Pat. Nos. 6,280,978; 7,094,399; 6,083,702; US Patent Publication Nos. US 2006-0234247 A1, and US 2006-0194317 A1, the contents of each of which are incorporated by referenced herein in their entirety) in an exemplary Fibroblast cell, although the method can be used with any dividing or non-dividing somatic cell.

The PTMs of the invention comprise a target binding domain that is designed to specifically bind to endogenous pre-mRNA, a 3′ splice region that includes a branch point, pyrimidine tract and a 3′ splice acceptor site and/or a 5′ splice donor site; and a spacer region that separates the RNA splice site from the target binding domain. In addition, the PTMs of the invention can be engineered to contain any nucleotide sequences encoding a pluripotency factor(s), which upon trans-splicing, produces a functional pluripotency factor that causes induction of the non-pluripotent cell to a pluripotent stem cell. In a preferred embodiment, the pluripotency factor translated upon trans-splicing using SMaRT™ induces the transformation of the non-pluripotent cell (i.e. reprogramming of a cell). The methods of the invention encompass contacting the PTMs of the invention with a natural endogenous pre-mRNA under conditions in which a portion of the PTM is trans-spliced to a portion of the natural endogenous pre-mRNA to form a novel chimeric mRNA. Specificity can be achieved by modification of the binding domain of the PTM to bind to the target endogenous pre-mRNA.

The PTMs of the invention thus comprise (i) one or more target binding domains that target binding of the PTM to a pre-mRNA (ii) a 3′ splice region that includes a branch point, pyrimidine tract and a 3′ splice acceptor site and/or 5′ splice donor site; and (iii) a spacer region to separate the RNA splice site from the target binding domain. Additionally, as described above, the PTMs are engineered to contain any nucleotide sequence encoding a pluripotency factor(s), which upon trans-splicing, produces a functional pluripotency factor that causes induction of the non-pluripotent cell to a pluripotent stem cell.

The target binding domain of the PTM may contain one or two binding domains of at least 15 to 30; or having long binding domains as described in US Patent Publication No. US 2006-0194317 A1 (the contents of which are incorporated herein by reference in their entirety), of up to several hundred nucleotides which are complementary to and in anti-sense orientation to the targeted region of the selected endogenous pre-mRNA. This confers specificity of binding and anchors the endogenous pre-mRNA closely in space so that the spliceosome processing machinery of the nucleus can trans-splice a portion of the PTM to a portion of the endogenous pre-mRNA. A second target binding region may be placed at the 3′ end of the molecule and can be incorporated into the PTM of the invention. Absolute complementarity, although preferred, is not required. A sequence “complementary” to a portion of the endogenous pre-mRNA, as referred to herein, means a sequence having sufficient complementarity to be able to hybridize with the endogenous pre-mRNA, forming a stable duplex. The ability to hybridize will depend on both the degree of complementarity and the length of the nucleic acid (See, for example, Sambrook et al., 1989, Molecular Cloning, A Laboratory Manual, 2d Ed., Cold Spring Harbor Laboratory Press, Cold Spring Harbor, N.Y.). Generally, the longer the hybridizing nucleic acid, the more base mismatches with an RNA it may contain and still form a stable duplex. One skilled in the art can ascertain a tolerable degree of mismatch by use of standard procedures to determine the melting point of the hybridized complex.

Binding may also be achieved through other mechanisms, for example, through triple helix formation or protein/nucleic acid interactions such as those in which the PTM is engineered to recognize a specific RNA binding protein, i.e., a protein bound to a specific target endogenous pre-mRNA. Alternatively, the PTMs of the invention may be designed to recognize secondary structures, such as for example, hairpin structures resulting from intramolecular base pairing between nucleotides within an RNA molecule.

The PTM molecule also contain a 3′ splice region that includes a branch point, pyrimidine tract and a 3′ splice acceptor AG site and/or a 5′ splice donor site. Consensus sequences for the 5′ splice donor site and the 3′ splice region used in RNA splicing are well known in the art (See, Moore, et al., 1993, The RNA World, Cold Spring Harbor Laboratory Press, p. 303-358). In addition, modified consensus sequences that maintain the ability to function as 5′ donor splice sites and 3′ splice regions may be used in the practice of the invention. Briefly, the 5′ splice site consensus sequence is AG/GURAGU (where A=adenosine, U=uracil, G=guanine, C=cytosine, R=purine and/=the splice site). The 3′ splice site consists of three separate sequence elements: the branch point or branch site, a polypyrimidine tract and the 3′ consensus sequence (YAG). The branch point consensus sequence in mammals is YNYURAC (Y=pyrimidine). The underlined A is the site of branch formation. A polypyrimidine tract is located between the branch point and the splice site acceptor and is important for branch point utilization and 3′ splice site recognition.

A spacer region to separate the RNA splice site from the target binding domain is also included in the PTM. The spacer region can have features such as stop codons which would block any translation of an unspliced PTM and/or sequences that enhance trans-splicing to the target pre-mRNA.

In a preferred embodiment of the invention, a “safety” design of the binding domain is also incorporated into the spacer, binding domain, or elsewhere in the PTM to prevent non-specific trans-splicing. The spacer sequence is a region of the PTM that covers elements of the 3′ and/or 5′ splice site of the PTM by relatively weak complementarity thereby preventing non-specific trans-splicing. The PTM is designed in such a way that upon hybridization of the binding/targeting portions) of the PTM, the 3′ and/or 5′splice site is uncovered and becomes fully active. The “safety” sequence consists of one or more complementary stretches of cis-sequence (or could be a second, separate, strand of nucleic acid) which weakly binds to one or both sides of the PTM branch point, pyrimidine tract, and/or 3′ splice site (splicing elements), or could bind to parts of the splicing elements themselves. This “safety” sequence binding prevents the splicing elements from being active (i.e. block U2 snRNP or other splicing factors from attaching to the PTM splice site recognition elements). The binding of the “safety” sequence may be disrupted by the binding of the target binding region of the PTM to the target pre-mRNA, thus exposing and activating the PTM splicing elements (making them available to trans-splice into the target endogenous pre-mRNA).

Additional features can be added to the PTM molecule either after, or before, the nucleotide sequence encoding a translatable protein, such as polyadenylation signals or 5′ splice sequences to enhance splicing, additional binding regions, “safety” sequence self-complementary regions, additional splice sites, or protective groups to modulate the stability of the molecule and prevent degradation. Additional features that may be incorporated into the PTMs of the invention include stop codons or other elements in the region between the binding domain and the splice site to prevent unspliced pre-mRNA expression. In another embodiment of the invention, PTMs can be generated with a second anti-sense binding domain downstream from the nucleotide sequences encoding a translatable protein to promote binding to the 3′ target intron or exon and to block the fixed authentic cis-5′ splice site (U5 and/or U1 binding sites). PTMs may also be made that require a double trans-splicing reaction for expression of the trans-spliced product. Such PTMs could be used to replace an internal exon which could be useful for RNA repair. Further elements such as a 3′ hairpin structure, circularized RNA, nucleotide base modification, or a synthetic analog can be incorporated into PTMs to promote or facilitate nuclear localization and spliceosomal incorporation, and intracellular stability.

The PTMs of the invention can be used in methods designed to produce a novel chimeric mRNA in a target cell such as, for example, a somatic cell. The methods of the present invention comprise delivering to the target cell a PTM which may be in any form used by one skilled in the art, for example, an RNA molecule, an RNA vector or a DNA vector which is transcribed into a RNA molecule, wherein the PTM binds to an endogenous pre-mRNA and mediates a trans-splicing reaction resulting in formation of a chimeric RNA comprising a portion of the PTM molecule spliced to a portion of the endogenous pre-mRNA.

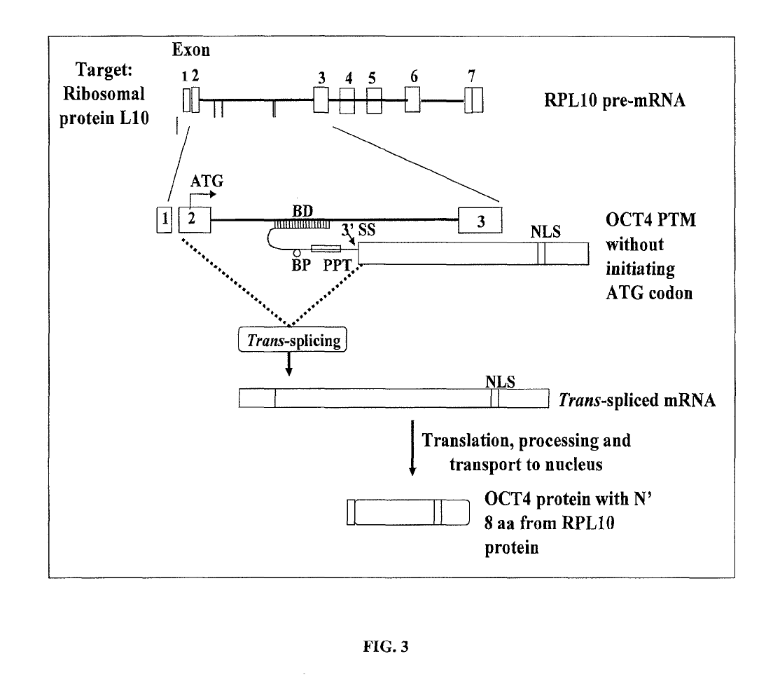

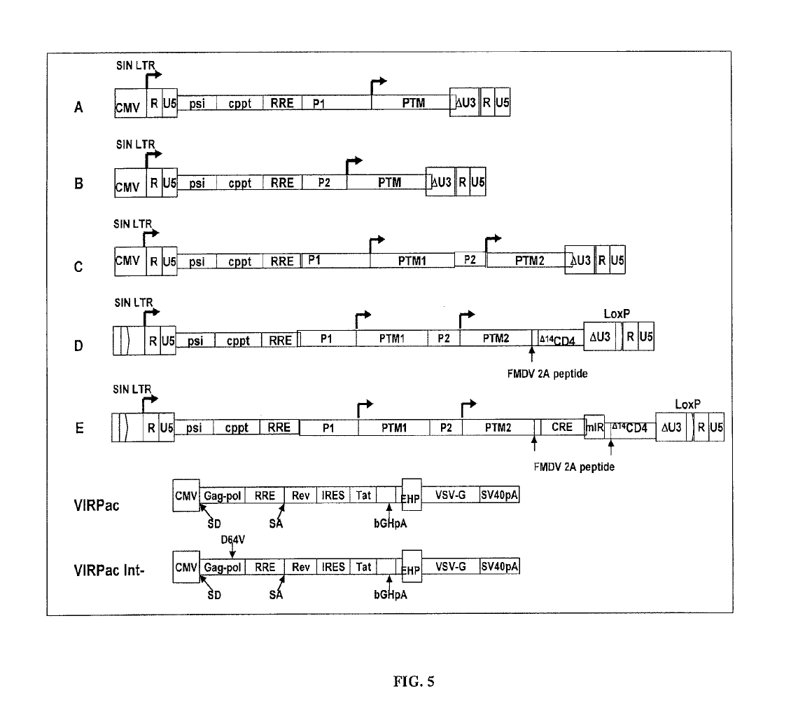

FIG. 3 shows an example of one of the transcription factors (OCT3/4), trans-spliced into Ribosomal protein L10 pre-mRNA target. The activity of OCT3/4 would be under endogenous regulation of the chosen target gene(s) since the PTMs cannot produce functional pluripotency factors in the absence of trans-splicing with the endogenous targeted pre-mRNA transcripts. PTMs can be delivered using viral vectors (e.g., lentiviral, Adeno-associated viral (“AAV”), Adenoviral, EBV, HSV, Rabies, hybrid vectors comprising AAV and Lentiviral vector, etc.) or non-viral vectors (e.g. plasmid DNA vectors including, for example, minicircle DNA vectors, (Chen et al., Hum Gene Ther 16, 126-131, 2005), transposon delivery systems, phage, or PTM RNA molecules. Furthermore, the expression of the PTMs can be regulated by a constitutive promoter(s) or an inducible promoter(s) or a tissue specific promoter(s) or their combination, and may be bidirectional, capable of driving the expression of one or more different PTMs in a single vector (FIG. 5).

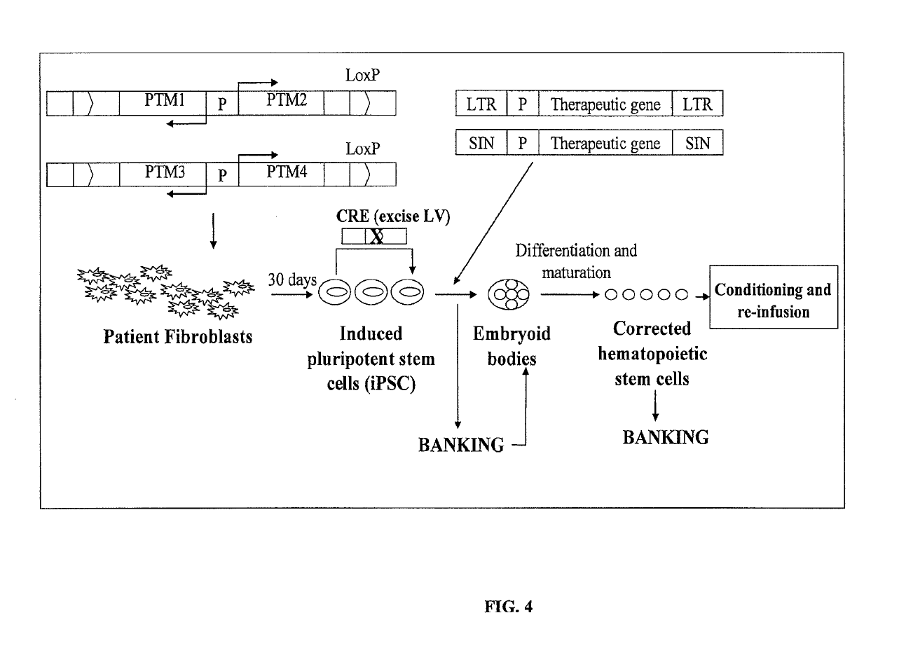

FIG. 4 is a schematic diagram showing the use of SMaRT™ technology to reprogram non-pluripotent cells to induced pluripotent stem cells and for the therapeutic correction of diseased induced pluripotent stem cells [P, promoter; PTM, pre-trans-splicing molecule; SIN, self inactivating LTR]. FIG. 4 describes reprogramming of differentiated human non-pluripotent cells to a pluripotent stage permits the establishment of patient disease-specific induced pluripotent stem cells via the delivery of PTMs by lentiviral vectors. These iPSC can then be repaired using RNA trans-splicing to correct a genetic defect. Accordingly, introduction can be effected, for instance, in vitro (e.g., in an ex vivo type method), which includes the use of electroporation, transformation, transduction, conjugation or transfection, infection, membrane fusion with cationic lipids, high-velocity bombardment with DNA-coated microprojectiles, incubation with calcium phosphate-DNA precipitate, direct microinjection into single cells, and the like. Other methods also are available and are known to those skilled in the art.

Thus, the present invention describes the use of SMaRT™ technology to produce different combinations of transcription factors in patient specific somatic cells. This is achieved by trans-splicing PTMs encoding these pluripotency factors into one or more endogenous pre-mRNAs in somatic cells. The target pre-mRNA transcripts can include those that are constitutively expressed or that are down regulated after a pluripotent has been achieved.

The PTMs can therefore be designed with different binding domains and coding domains to target defective pre-mRNA for repair or to modify highly expressed pre-mRNAs to generate therapeutic proteins of interest or image gene expression for diagnostic applications. Trans-splicing between the PTM and target pre-mRNA may not occur until the pre-mRNA is expressed, which may be critical for some applications where early expression of a transgene may be detrimental to the cell, e.g. expression of cystic fibrosis transmembrane conductance regulator protein (CFTR) in pulmonary stem cells.

In one embodiment of the invention, the transduced somatic cells are cultured until embryonic stem cell like colonies are observed. The colonies are picked and expanded in defined media under feeder-free conditions or on a human feeder cell layer. On reprogramming of the somatic cells into pluripotent stem cells, the pluripotency transcription factors will be silenced or down regulated. Alternatively, the genes or PTMs can be excised, e.g. by incorporating Lox-sites into integrating vectors and expressing Cre-recombinase, or silenced, e.g. by incorporating sequence(s) targeted by stage (lineage-, tissue-)-specific siRNA or micro-RNA, as an additional safety measure (FIG. 4).

After several passages to ensure selection and purity of the iPSC population, the iPSC can be transduced with different PTM types depending on the application using viral vectors or non-viral vectors. At this stage the transduced iPSC can either be stored in a cell bank or will be used directly to form embryoid bodies, the precursor state from which several different cell types can be created using lineage-specific growth signals including for example, cytokines, chemokines, and any other factors required for lineage specific differentiation as specified supra. Day 7 or older transduced embryoid bodies can be differentiated in several cell types including cells of blood, endothelium and neural cells. The matured/differentiated cells now carrying the therapeutic gene can either be stored in a cell bank and/or can then be re-infused back into a patient (FIG. 4).

### Methods of Use

As described supra, the present invention also relates to methods for creation of iPSC that are derived by reprogramming a non-pluripotent cell to a pluripotent stem cell using SMaRT™. The reprogramming step involves the introduction of at least one PTM encoding a non-functional pluripotency factor(s) into a non-pluripotent cell, which upon trans-splicing, produces a functional mRNA transcript that is translated into a protein causing induction of the reprogramming/de-differentiation of the non-pluripotent cell into a pluripotent stem cell. The reprogramming step may also be carried out with diseased non-pluripotent cells using at least one PTM encoding a pluripotency factor(s) in combination with a corrective gene product(s), which upon trans-splicing, produces a functional factor that causes induction of the non-pluripotent cell to a pluripotent stem cell in which the diseased phenotype has been corrected.

Thus, in one aspect of the present invention, a method is therefore provided for creation of induced pluripotent stem cells comprising introducing into a non-pluripotent cell at least one PTM encoding a pluripotency factor(s), which upon trans-splicing using spliceosome-mediated RNA trans-splicing, produces a functional factor that causes induction of the non-pluripotent cell to a pluripotent stem cell.

In one particular embodiment of the method of the present invention, the pluripotency factors encoded by the at least one PTM can be a transcription factor of an OCT family gene, a KLF family gene, a MYC family gene, or a SOX family gene, or any combination thereof.

In another particular embodiment, the present invention also provides such a method wherein the transcription factor can comprise one or more gene products of each of: an OCT family gene, a KLF family gene, and a SOX family gene, or any combination thereof.

In another particular embodiment, the present invention also provides such a method wherein the transcription factor can comprise one or more gene products of each of: an OCT family gene, a KLF family gene, a MYC family gene, and a SOX family gene.

In another particular embodiment, the present invention also provides such a method wherein the transcription factor can further comprise one or more gene products of a SALL4 gene.

In another particular embodiment, the present invention also provides such a method wherein the pluripotency factor comprises one or more gene products of each of: OCT3/4, KLF4, c-MYC, and SOX2.

In another particular embodiment, the present invention also provides such a method wherein the transcription factor comprises one or more gene products of each of: KLF4, c-MYC, OCT3/4, SOX2, NANOG, and LIN28.

In another particular embodiment of the method of the present invention, the transcription factor can comprise at least one of OCT3/4, SOX2, KLF4, c-MYC, NANOG, LIN 28, UTF1, p53 siRNA or any combination thereof.

Furthermore, in another particular embodiment, the present invention also provides such a method wherein the transcription factor can comprise one or more gene products of each of: an OCT family gene, a KLF family gene, in combination with a cytokine.

In each instance, the present invention also provides an induced pluripotent stem cell obtained by any of the aforementioned methods.

In another aspect of the invention, a method is provided for creation of induced pluripotent stem cells comprising introducing into a non-pluripotent cell at least one first PTM encoding a pluripotency factor(s), and at least one second PTM encoding a therapeutic gene product(s), which upon trans-splicing using SMaRT™, produces a functional factor that causes induction of the non-pluripotent cell to a pluripotent stem cell; and whereupon trans-splicing of the second PTM using SMaRT™ results in expression of said therapeutic gene product.

In one embodiment of the invention, a method is provided for creation of induced non-diseased pluripotent stem cells comprising introducing into a diseased non-pluripotent cell at least one first PTM encoding a pluripotency factor(s), and at least one second PTM encoding a therapeutic gene product(s), which upon trans-splicing using SMaRT™, produces a functional factor that causes induction of the non-pluripotent cell to a pluripotent stem cell; and whereupon trans-splicing of the second PTM using SMaRT™ results in expression of said therapeutic gene product so as to generate a non-diseased pluripotent stem cell in which the therapeutic gene product is expressed.

In one particular embodiment of the method of the present invention, the at least one first PTM encoding a pluripotency factor(s) and the at least one second PTM encoding a therapeutic gene product(s) are co-expressed from the same vector, which upon trans-splicing using SMaRT™, produces a functional factor that causes induction of the non-pluripotent cell to a pluripotent stem cell in which the therapeutic gene product(s) are expressed.

In yet another particular embodiment of the method of the present invention, the at least one first PTM encoding a pluripotency factor(s) and the at least one second PTM encoding a therapeutic gene product(s) are expressed from separate vectors delivered either separately in any order or at the same time, which upon trans-splicing using SMaRT™, produces a functional factor that causes induction of the non-pluripotent cell to a pluripotent stem cell in which the therapeutic gene product(s) are expressed.

In yet another particular embodiment of the method of the present invention, a diseased non-pluripotent cell may be reprogrammed to an induced pluripotent stem cell using one or more of the aforementioned pluripotency factor(s). Therapeutic genes using PTMs can then be delivered using SMaRT™ to the reprogrammed, but still diseased pluripotent stem cells, such that SMaRT™ will re-program defective transcripts only when the specific targeted endogenous pre-mRNA is expressed in progeny cells.

In yet another aspect, the present invention also provides for the generation of somatic cells derived by inducing differentiation of the aforementioned induced pluripotent stem cells. The present invention thus provides a somatic cell derived by inducing differentiation of the aforementioned induced pluripotent stem cells.

Thus, in one particular embodiment, the present invention comprises a method for stem cell therapy in a patient comprising: isolating and collecting a non-pluripotent cell from a patient; inducing said non-pluripotent cell from the patient into an induced pluripotent stem cell by introducing at least one PTM encoding a pluripotency factor(s), which upon trans-splicing using SMaRT™, produces a functional factor that causes induction of the non-pluripotent cell to a pluripotent stem cell; and then inducing lineage commitment/differentiation of said induced pluripotent stem cell, and transplanting resulting differentiated cell into said patient. A non-limiting example of the application of this method would be use of SMaRT™ to generate a patient specific iPS cell line to further generate hematopoietic stem cells for transplantation when the patient does not have compatible MHC donors and is in dire need of a transplantation following treatment, for example, of cancer.

The present invention also provides for the generation of somatic cells or non-pluripotent fully differentiated cells derived by inducing differentiation of the aforementioned induced pluripotent stem cells obtained by any of the aforementioned methods.

### Pharmaceutical Compositions

The pharmaceutical compositions of the present invention contain a pharmaceutically and/or therapeutically effective amount of a patient's reprogrammed cells derived from iPS cells generated by any of the aforementioned vectors expressing one or more of the pluripotency reprogramming factor(s) encoded by the at least one PTM. In one embodiment, the effective amount of a pluripotency factor(s) encoded by the at least one PTM per unit dose is an amount sufficient to produce, upon trans-splicing using SMaRT™, a functional factor that causes induction of the non-pluripotent cell to a pluripotent stem cell. In another embodiment of the invention, the effective amount of a pluripotency factor(s) encoded by the at least one PTM in combination with a corrective gene product(s), is an amount, which upon trans-splicing, produces a functional factor that causes induction of the non-pluripotent cell to a pluripotent stem cell, and which is sufficient to prevent, treat or protect against deleterious effects (including severity, duration, or extent of symptoms) of the disease or condition being treated.

The administration of the pharmaceutical compositions resulting from the compositions and methods of the invention may be for either “prophylactic” or “therapeutic” purpose. When provided prophylactically, the compositions are provided in advance of any symptom. The prophylactic administration of the composition serves to prevent a disease or condition. When provided therapeutically, the composition is provided at (or shortly after) the onset of a symptom of the condition being treated.

In yet another embodiment of the present invention, for all therapeutic, prophylactic and diagnostic derived uses, one or more of the aforementioned vectors, lentiviral vectors, lentiviral vector systems, or viral particle/virus stock of the present invention, as well as other necessary reagents and appropriate devices and accessories, may be provided in kit form. Such a kit would comprise a pharmaceutical composition for ex vivo, in vitro (or if applicable, in vivo) administration comprising a vector, lentiviral vectors, lentiviral vector systems, or viral particle/virus stock containing one or more of the pluripotency factor(s) of the present invention encoded by the at least one PTM, and a pharmaceutically acceptable carrier and/or a genetic adjuvant; and instructions for use of the kit.

Pharmaceutical formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers. Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials. Preferred unit dosage formulations are those containing a dose or unit, or an appropriate fraction thereof, of the administered ingredient. It should be understood that in addition to the ingredients, particularly mentioned above, the formulations may also include other agents commonly used by one of ordinary skill in the art.

The pharmaceutical formulation comprising cells derived from the subject's specific iPSC generated using the compositions and methods of the present invention may be administered through different routes, such as oral, including buccal and sublingual, rectal, parenteral, aerosol, intranasal, intramuscular, subcutaneous, intravenous, intraperitoneal, intraocular, intracranial, intradermal, topical, or direct injection into a joint or other area of the subject's body or any combination thereof. The autologous cells or transplantable cells generated from the subject's specific iPSC generated may likewise be administered in different forms, including but not limited to solutions, emulsions and suspensions, microspheres, particles, and microparticles. An appropriate quantity of the subject's specific cells derived from iPSC to be administered is determined for any of the methods disclosed herein by one skilled in the art based on a variety of physical characteristics of the subject or patient, including, for example, the patient's age, body mass index (weight), gender, health, immunocompetence, and the like. Similarly, the volume of cells administered will vary depending on the route of administration. By way of example, intramuscular injections may range from about 0.1 mL to 1.0 mL. Cell concentrations will also vary depending on the derived cell type and the cryo-preservation process for optimal post-thaw recovery before transplantation back to the subject.

The pharmaceutical preparation may be stored at temperatures of from about −80° C. to about 37° C. or less, depending on how the pharmaceutical preparation is formulated. A variety of adjuvants known to one of ordinary skill in the art may be administered in conjunction with the reprogrammed iPSC in the pharmaceutical composition.

The invention now being generally described, it will be more readily understood by reference to the following examples, which are included merely for purposes of illustration of certain aspects and embodiments of the present invention, and are not intended to limit the invention. The contents of any patents, patent applications, patent publications, or scientific articles referenced anywhere in this application are herein incorporated in their entirety.

### EXAMPLES

### Example 1

### RNA Trans-Splicing Via Smart™

SMaRT™ involves trans-splicing between two RNA molecules: a pre-mRNA target endogenously expressed by a cell and an introduced RNA called a PTM (FIG. 2). PTMs are engineered RNA molecules that contain three functional domains: 1) A binding domain (BD) anchors the PTM to a selected intron region which serves as target, 2) a splicing domain with conserved elements that are required for efficient splicing, and 3) a coding region consisting of one or more exons of the cDNA encoding for a transcript to be trans-spliced. An important safety feature of trans-splicing technology is that the coding region of a PTM cannot be translated into a functional protein unless it is trans-spliced into an available endogenous target pre-mRNA. For iPSC derivation, PTMs for pluripotency factors will be trans-spliced into endogenous pre-mRNA targets that are preferentially expressed in non-pluripotent cells but not in iPSC. As the cells re-program into iPSC, the endogenous pre-mRNA targets will be down-regulated or shut off resulting in cessation of trans-splicing and no production of transcription factors. This important safety feature will ensure that there will be no re-activation of potentially oncogenic transcription factors such as c-MYC or KLF4 once re-programming into iPSC is complete.

### Example 2

### Strategy for PTM Based iPS Cell Generation

A specific example of a pre-mRNA target that can be used for trans-splicing of transcription factor PTMs is the RPL10 gene. RPL10 is expressed at about 5 times higher levels in fibroblasts non-pluripotent cells as compared to iPSC. This makes it a potential target that can be used to trans-splice transcription factor PTMs into. The transcription factor OCT3/4 PTM is constructed to contain a binding domain (BD) targeting RPL10 intron 2, a branch point (BP), a poly pyrimidine tract (PPT), a 3′ splice site (SS) and the OCT3/4 coding region lacking an initiating ATG codon. Trans-splicing of the OCT3/4 PTM will occur within the RPL10 pre-mRNA at intron 2 and will result in translation of OCT3/4 protein from the ATG initiating codon supplied by the RPL10 pre-mRNA (FIG. 3).

### Example 3

### Derivation of iPSC using PTMs Coding for Pluripotency Factors

Primary skin fibroblasts cultures are established from the patient's skin biopsy sample and cultured for about 7 to about 14 days. 5×10⁴ primary fibroblast are transduced overnight with a combination of four lentiviral vectors containing the PTMs for OCT3/4, SOX2, NANOG, LIN28 or OCT3/4, SOX2, KLF4, c-MYC or a combination thereof, at 2.5×10⁵ to 5×10⁵ transducing units of each vector, for 12-16 hr. Alternatively, by using bidirectional constitutive or tissue specific promoters, each vector will express two of the PTMs. The lentiviral vectors also have LoxP sites flanking the PTMs which can later be excised with Cre recombinase enzyme expressing vector, which provides an additional safety feature that ensures removal of the vector after reprogramming to the pluripotent stem cell stage is achieved. In an alternative strategy, the PTMs will also be delivered using IDLV vectors that will have impaired ability to integrate into the target cell genome. The transduced fibroblasts are cultured for about 6 days in DMEM with 10% serum followed by plating 5×10⁴ cells per 100 mm dish on feeder cell layer. The cells are cultured for an additional 30 days in human ES media containing 4 ng/ml basic fibroblast growth factor (bFGF). iPSC colonies are isolated and expanded on feeder cells or in feeder free conditions on Matrigel coated plates. The colonies are transduced with a Cre expressing plasmid system to excise the PTMs, and then expanded for 5-10 passages. About 10 to about 20 million iPSC are frozen down and banked. These patient specific iPSC will be transduced with lentiviral vectors coding for PTMs to correct for the genetic defect or to express therapeutic proteins. Specific examples of the use of SMaRT™ derived IPSC are detailed below.

### Example 4

### Strategy for the Treatment of Cystic Fibrosis

Cystic fibrosis gene therapy research involving stem cells has shown that ectopic CFTR expression in airway stem cells confers a selective disadvantage to reconstitution and persistence of epithelium in human airways. Similarly, overexpression of CFTR in lung epithelial cells or heterologous expression in other cell types is detrimental to cell physiology (Schiavi et al., Am J Physiol 270, C341-351, 1996; Mohammad-Panah et al., Am J Physiol 274, C310-318, 1998; Stuffs et al., J Biol Chem 268, 20653-58). A solution to this problem is described herein.

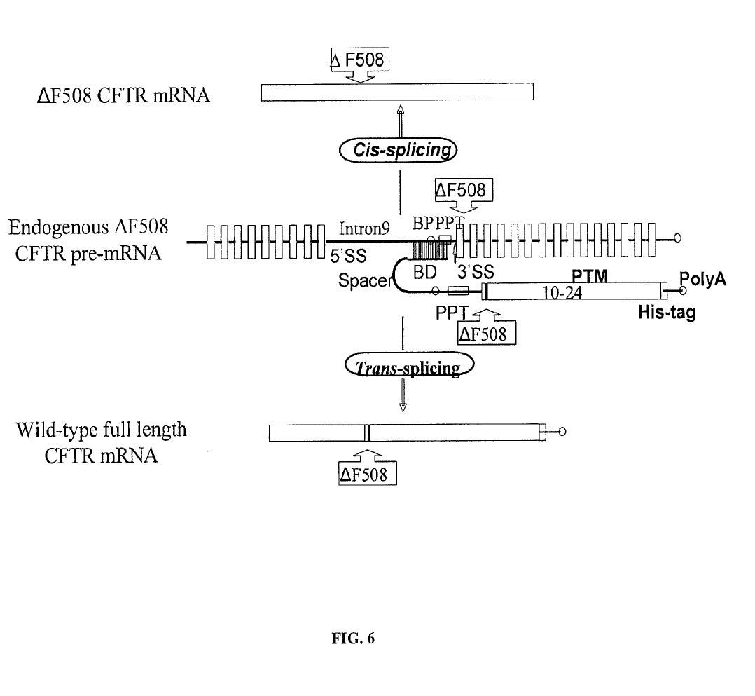

SMaRT™ based repair strategy described here does not permit expression of normal CFTR proteins until the differentiating stem cell begins to naturally express mutant CFTR transcripts, and eliminates the selective disadvantage in corrected stem cells. CFTR transcripts having the most common mutation (ΔF508 in exon 10) can be repaired using a 3′ exon replacement PTM with a single binding domain targeted to the 3′ end of intron 9 in CFTR pre-mRNA and designed to replace exons 10 through 24. This PTM was shown to correct the major mutation for cystic fibrosis (ΔF508 in exon 10) and any mutation distal to this (FIG. 6). SMaRT™ based repair allows each cell to produce its own prescribed amount of CFTR proteins. Repair of CFTR pre-mRNA can also be performed by 5′ exon replacement where the PTM contains exons 1 to 10 and a binding domain targeted to intron 10. In this case the PTM would trans-splice to CFTR pre-mRNA in exon 11.

For the correction of CFTR defect, SMaRT™ derived patient iPSC are transduced with lentiviral vectors expressing the CFTR PTM. Alternatively, the CFTR PTM can also be delivered into the patient fibroblasts along with the pluripotency factor PTM's during the re-programming process (FIG. 1). The CFTR PTM will not be trans-spliced until the endogenous mutant CFTR pre-mRNA transcripts are produced at the appropriate airway epithelial differentiation stage. The iPSC transduced with the corrected CFTR PTM will be differentiated into airway epithelial stem cells and transplanted into the patient's airway.

### Example 5

### Strategy for the Treatment of Hemophilia A

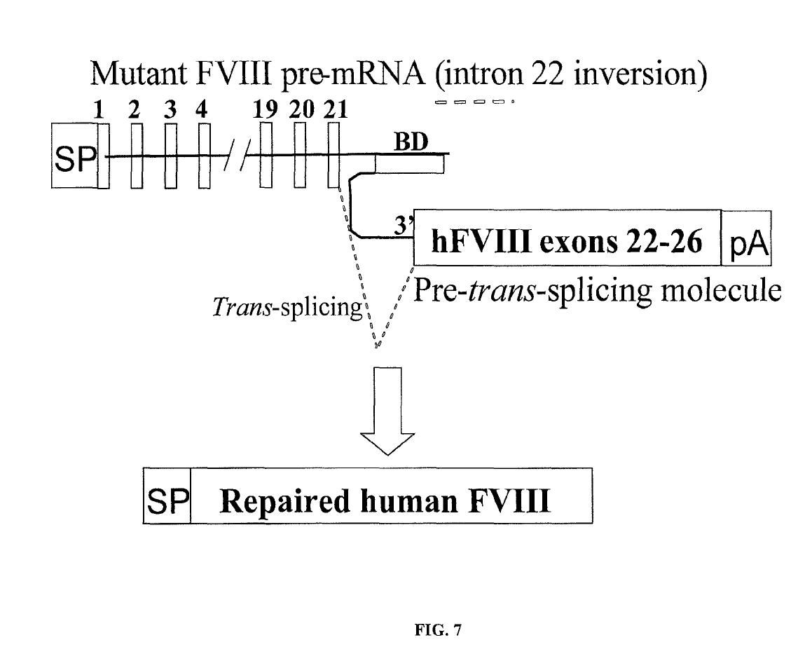

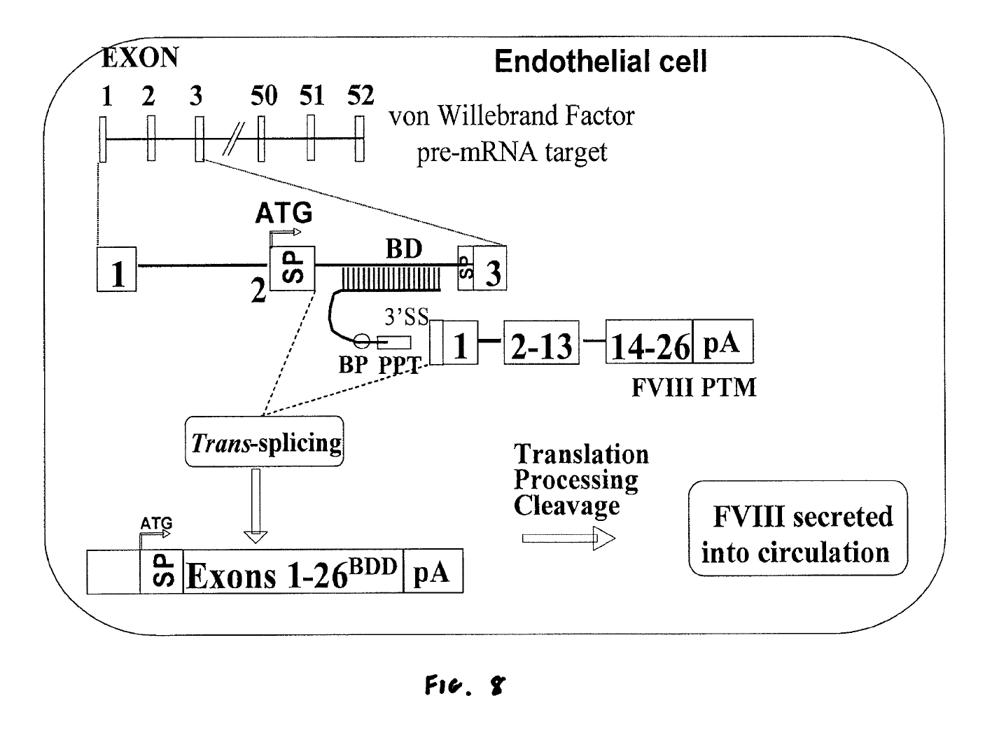

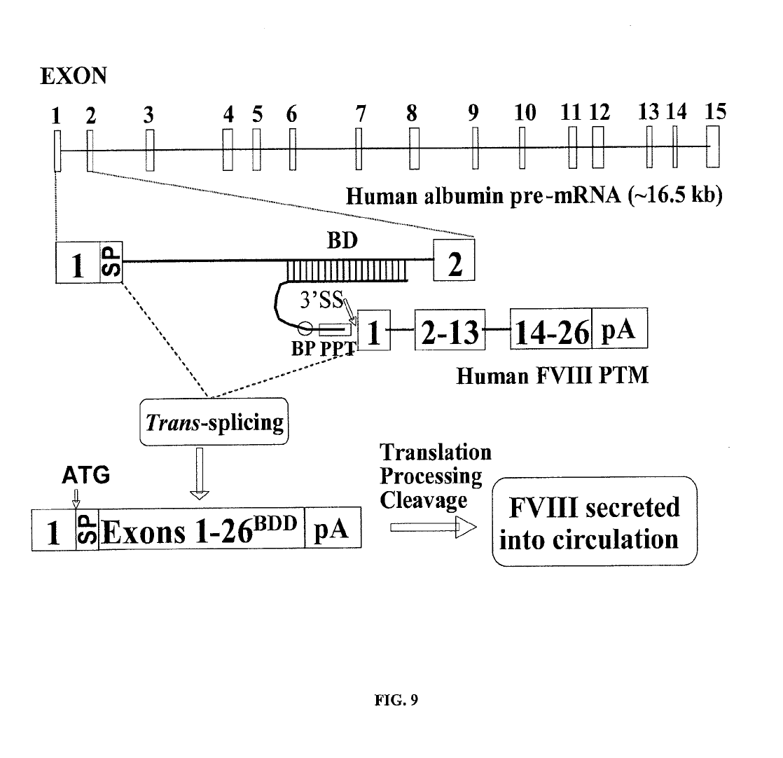

Hemophilia A is an inherited bleeding disorder caused by a deficiency of blood coagulation factor VIII (FVIII) leading to frequent spontaneous intra-articular joint and soft tissue bleeding episodes. Since 5% of normal FVIII plasma concentration is therapeutic, hemophilia A is a prime disorder for genetic correction. Patients would benefit greatly from prophylactic therapy, and well characterized animal models are available for preclinical testing. Preclinical data demonstrates that somatic cell gene therapy can correct the disease in hemophilia A mice. Repair of defective human FVIII pre-mRNA can be achieved using a PTM containing exons 21 to 26 and with a single binding domain targeted to intron 21 in FVIII pre-mRNA (FIG. 7). This PTM will correct >60% of the mutations responsible for hemophilia A including the most prevalent mutation, the intron 22 inversion mutation. Repair of FVIII can be achieved by transducing SMaRT™ derived iPSC with a FVIII PTM targeted to highly abundant transcripts in endothelial cells e.g. von Willebrand Factor (vWF). Secreted FVIII can be generated using a PTM that is targeted to intron 2 of vWF and that contains exons 1 to 26 of human FVIII (minus the FVIII signal peptide) (FIG. 8). Trans-splicing of a human FVIII PTM to any human pre-mRNA to create high levels of circulating FVIII can be achieved using a human FVIII PTM containing exons 1 to 26 (minus the signal peptide) targeted to intron 1 of any human pre-mRNA that contains a signal peptide sequence for protein secretion. Trans-splicing between the PTM and the selected target will produce secreted FVIII (FIG. 9). This approach can be used to treat >95% of hemophilia A patients. Endothelial cells will be derived by differentiating iPSC using specific culture conditions that promote endothelial differentiation. These corrected cells will be transplanted into the patient.

The PTM can be adapted to repair any and all mutations in the FVIII gene.

### Example 6

### Strategy for the Treatment of Tau-Related Neurodegenerative Diseases

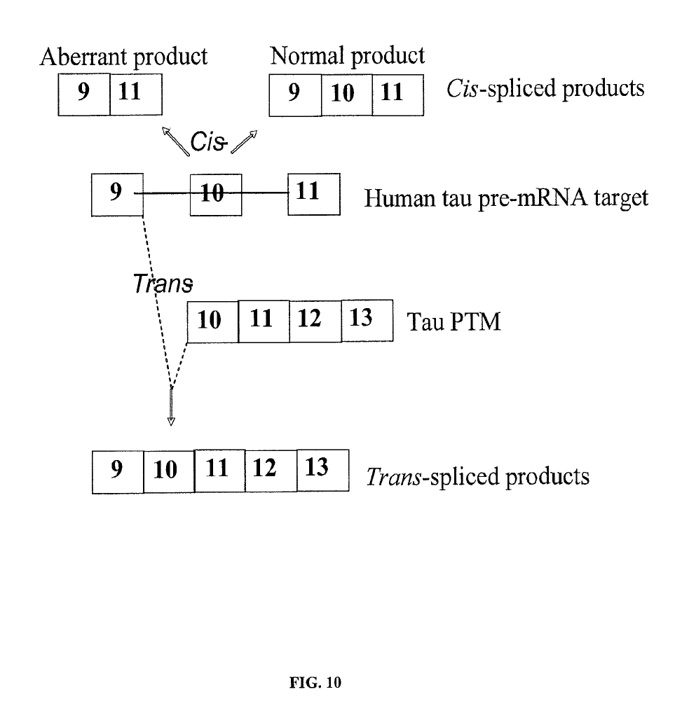

Mutations in the microtubule-associated protein tau can lead to frontotemporal dementias. Some of these dementias are caused by exon 10 skipping and this aberrant splicing can potentially be corrected with reprogramming of tau alternative splicing. This can be achieved with a PTM containing human tau exons 10 to 13 and a binding domain complementary to the 3′ end of tau intron 9 (FIG. 10). The related therapy would encompass the concept of generating subject specific iPSC with the present invention and generating corrected neural stem cells with the PTM described above.

### Example 7

### Strategy for the Treatment of Hypercholesterolemia

Cardiovascular disease (CVD) is the most common cause of death in Western societies. One of the strongest predictors of risk is the plasma concentration of high-density lipoprotein (HDL) or apolipoprotein A1 (apoA1), the major protein component of HDL, which exhibits an inverse relationship with the development of atherosclerosis and coronary heart disease. ApoA-1 is the major apolipoprotein of HDL and plays an important role in promoting the efflux of excess cholesterol from peripheral cells and tissues for transfer to the liver for excretion, a process called reverse cholesterol transport (RCT).

Numerous in vitro and in vivo studies have demonstrated the protective effects of apoA1 and HDL against atherosclerosis plaque development. ApoA-1 deficiency and/or low HDL cholesterol conditions can be treated by targeting “highly abundant transcripts” such as albumin pre-mRNA or more likely macrophage specific pre-mRNA to increase the level of human apoA-I protein and HDL. This can be achieved using a PTM with a single binding domain targeted to intron 1 in the selected pre-mRNA and designed to contain majority of the human apoA-I coding domain. iPSC derived macrophage or hepatocytes expressing apoA-I will be infused or transplanted. This strategy could also be expanded to include other dyslipidemia targets such as lecithin-cholesterol acyltransferase (LCAT), ATP-binding cassette, sub-family A (ABC1), low density lipoprotein receptor (LDLR) etc.

### Example 8

### Strategy for the Treatment of ATT Deficiencies

Correction of AAT deficiency, and its associated lung and liver pathology (FIG. 10), can be repaired by correction of PI-ZZ mutation in alpha1 anti-trypsin (AAT) deficiency, to replace either exons 2-5 or 3-5, depending on the mutations. This can be achieved in three distinct mechanisms to reduce the lung and liver pathology of PI-ZZ patients: (1) PTM can be designed to replace the defective exon 5 in the PI-ZZ Serpina1 pre-mRNA with a normal exon 5 sequence leading to correction of the defective mRNA sequence and providing synthesis of normal AAT protein, thereby elevating blood levels of normal AAT protein and reduce AAT lung disease, (2) PTMs containing the entire AAT mature coding sequence can be used to target pre-mRNAs to produce functional AAT protein, thereby elevating blood levels of normal AAT protein and reduce AAT lung disease. These two approaches encompass the concept of modifying iPSC derived blood cells or even hepatocytes followed by transfusion or transplantation. In the later case, the trans-splicing reaction could also reduce the abnormal pre-mRNA present in hepocytes as well, thus reducing the risk of developing liver disease.

### Example 9

### Strategy for the Treatment of Thalassemia and Sickle Cell Disease

Thalassemia is an inherited autosomal recessive blood disease. Thalassemia results in under production of globin proteins through mutations in globin genes. The severity of the disease depends on the nature of the mutation. Mutations in globin genes can be repaired using a PTM targeting specific mutation and/or a PTM with entire globin sequence can be trans-spliced to highly abundant transcript in the iPS derived hematopoietic cells.

### Example 10

### Strategy for Treatment of Type 1 Diabetes with iPSC Derived and Genetically Modified Pancreatic Endodermal Cells

For patients suffering from Type 1 diabetes or certain type 2 diabetes (insufficient insulin production) iPSC can be transduced to express human insulin and/or glucagon like peptide-1 (GLP-1) and then differentiated into pancreatic islet cells. iPSC are generated as described in example 1 and 2. iPSC are transduced with a lentiviral vector expressing the human insulin PTM and/or GLP-1 PTM. iPSC are induced to differentiate along the endoderm lineage into pancreatic endodermal cells using growth factors activin, Wnt, KAAD-cyclopamine, human fibroblast growth factor 10 (FGF-10), retinoic acid, γ secretase inhibitor, extendin, insulin growth factor 1 (IGF1), hepatocyte growth factor 1 (HFG1), FBS, and growth supplements. 1×10⁸-1×10⁹ insulin secreting cells/kg body weight or 10,000 islet equivalents/kg body weight are injected via a catheter inserted through the upper abdomen and guided to the pancreas via the hepatic portal vein. An alternate protocol involves growth and encapsulation of insulin secreting cells in Gelfoam and Matrigel or other suitable artificial biomembrane material, and implantation into the recipient. Implants are done either in subcutaneous adipose tissue or under the kidney capsule.

### Example 11

### Strategy for Treatment of Diabetes with iPSC Derived and Genetically Modified Pancreatic Endodermal Cells by an IDLV/AAV Hybrid System

iPSC generated from the patients using SMaRT™ will be transduced with an IDLV/AAV hybrid vector expressing human insulin PTM and/or GLP-1 PTM at a moi of 5-10, for 12-16 hrs. The vector will then be washed off and the cells will be allowed to grow for a further 48-72 hrs. in order to start expressing the gene. iPSC will be directed to differentiate along the endoderm lineage into pancreatic endodermal cells using growth factors activin, Wnt, KAAD-cyclopamine, human fibroblast growth factor 10 (FGF-10), retinoic acid, γ secretase inhibitor, extendin, insulin growth factor 1 (IGF1), hepatocyte growth factor 1 (HFG1), FBS, and growth supplements. 1×10⁸-1×10⁹ insulin secreting cells/kg body weight or 10,000 islet equivalents/kg body weight could be then injected via a catheter inserted through the upper abdomen and guided to the pancreas via the hepatic portal vein. An alternate protocol involves growth and encapsulation of insulin secreting cells in Gelfoam and Matrigel or other suitable artificial biomembrane material, and implantation into the recipient. Implants will be done either in subcutaneous adipose tissue or under the kidney capsule.

### Example 12

### Regeneration of Cardiac Muscle Using IPSC

Cardiovascular disease leading to myocardial infarctions was estimated to occur in over one million Americans in 2006. Although transplants for severely damaged hearts are successful in over 90% of the cases, matched donors are in short supply. The use of iPSC to generate cardiac muscle from the patient's skin or blood cells would allow patients to be transplanted with healthy cardiac muscle without waiting for donor hearts and without the risks of organ rejection.

iPSC from the patient will be cultured under conditions favoring their differentiation into cardiomyocytes either using the EB differentiation method, co-culture with endodermal cell lines or inclusion of factors that will improve cardiomyocytes differentiation. The cardiomyocytes will then be transplanted into the recipient's damaged heart tissue.

The foregoing description of some specific embodiments provides sufficient information that others can, by applying current knowledge, readily modify or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. In the drawings and the description, there have been disclosed exemplary embodiments and, although specific terms may have been employed, they are unless otherwise stated used in a generic and descriptive sense only and not for purposes of limitation, the scope of the claims therefore not being so limited. Moreover, one skilled in the art will appreciate that certain steps of the methods discussed herein may be sequenced in alternative order or steps may be combined. Therefore, it is intended that the appended claims not be limited to the particular embodiment disclosed herein.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims; or in the text itself; and, each of these documents or references (“herein-cited references”), as well as each document or reference cited in each of the herein-cited references (including any manufacturer's specifications, instructions, etc.), is hereby expressly incorporated herein by reference.

What is claimed is:

1. A non-pluripotent cell comprising at least one pre-trans-splicing molecule (PTM), which, upon trans-splicing using spliceosome-mediated RNA trans-splicing (SMaRT™), produces a functional pluripotency factor that induce the non-pluripotent cell into a pluripotent stem cell.

2. The non-pluripotent cell of claim 1, wherein said PTM further comprises: one or more target binding domains that targets binding of the PTM to an endogenous pre-mRNA; a 3′ splice region that includes a branch point, pyrimidine tract and a 3′ splice acceptor site and/or 5′ splice donor site; and a spacer region to separate the RNA splice site from the target binding domain.

3. The non-pluripotent cell of claim 1, wherein said pluripotency factor comprises at least one transcription factor.

4. The non-pluripotent cell of claim 3, wherein said transcription factor comprises a transcription factor gene product of an OCT family gene, a KLF family gene, a MYC family gene, or a SOX family gene.

5. The non-pluripotent cell of claim 4, wherein said transcription factor comprises at least one of OCT3/4, SOX2, KLF4, c-MYC, OCT3/4, NANOG, LIN 28, or any combination thereof.

6. The non-pluripotent cell of claim 4, wherein said transcription factor comprises at least one of OCT3/4, SOX2, KLF4, c-MYC, or any combination thereof.

7. The non-pluripotent cell of claim 4, wherein said transcription factor comprises at least one of OCT3/4, SOX2, NANOG, LIN 28, or any combination thereof.

8. A method for generating a pluripotent stem cell comprising introducing into a non-pluripotent cell at least one PTM encoding a pluripotency factor(s);
trans-splicing said at least one PTM encoding a pluripotency factor into an endogenous pre-mRNA using SMaRT™;
wherein trans-splicing of at least one PTM encoding a pluripotency factor(s) into an endogenous pre-mRNA produces a functional transcript which is then translated into a pluripotency factor that induces the non-pluripotent cell into a pluripotent stem cell.

9. The method of claim 8, further comprising the step of targeting binding of said PTM, wherein said PTM comprises one or more target binding domains that targets binding of the PTM to an endogenous pre-mRNA of the cell;
a 3′ splice region that includes a branch point, pyrimidine tract and a 3′ splice acceptor site and/or 5′ splice donor site; and a spacer region to separate the RNA splice site from the target binding domain.

10. The method of claim 8, wherein said pluripotency factor comprises at least one transcription factor.

11. The method of claim 8, wherein said transcription factor comprises a transcription pluripotency factor gene product of an OCT family gene, a KLF family gene, a MYC family gene, or a SOX family gene.

12. The method of claim 8, wherein said transcription factor comprises at least one of OCT3/4, SOX2, KLF4, c-MYC, OCT3/4, NANOG, LIN 28, or any combination thereof.

13. The cell of claim 8, wherein said transcription factor comprises at least one of OCT3/4, SOX2, KLF4, c-MYC, or any combination thereof.

14. A non-pluripotent cell comprising:
at least one first PTM encoding a pluripotency factor, and further comprising at least one second PTM encoding a therapeutic product, which upon trans-splicing of both the first PTM encoding the pluripotency factor and the second PTM encoding the therapeutic gene product, produce a functional pluripotency factor that induces the non-pluripotent cell into a pluripotent stem cell expressing said therapeutic product.

15. The non-pluripotent cell of claim 14, where said PTM comprises:
one or more target binding domains that target binding of the PTM to an endogenous pre-mRNA;
a 3′ splice region that includes a branch point, pyrimidine tract and a 3′ splice acceptor site and/or 5′ splice donor site; and a spacer region to separate the RNA splice site from the target binding domain.

16. The non-pluripotent cell of claim 14, wherein said gene product pluripotency factor comprises at least one transcription factor.

17. The cell of claim 16, wherein said transcription factor comprises a transcription pluripotency factor gene product of an OCT family gene, a KLF family gene, a MYC family gene, or a SOX family gene, or a combination thereof.

18. The cell of claim 17, wherein said transcription factor comprises at least one of OCT3/4, SOX2, KLF4, c-MYC, NANOG, LIN 28, or any combination thereof.

19. The cell of claim 14, wherein the at least one second PTM encodes a therapeutic product enabling the repair or reprogrammation of pre-mRNAs for the correction of diseases such as cystic fibrosis, hemophilia, hypercholesterolemia, alpha1 anti-trypsin defiencies, Thalassemia, sickle cell, and diabetes among others.

20. A method for repairing a non-pluripotent cell comprising
introducing into a non-pluripotent cell at least one first PTM encoding a pluripotency factor;
introducing at least one second PTM encoding a therapeutic product;
trans-splicing said at least one first PTM encoding a pluripotency factor into an endogenous pre-mRNA of the non-pluripotent cell using SMaRT™;
trans-splicing said at least one second PTM encoding a therapeutic product into an endogenous pre-mRNA of the non-pluripotent cell using SMaRT™;
wherein trans-splicing of at least one PTM encoding a pluripotency factor(s) into an endogenous pre-mRNA and trans-splicing of at least one second PTM encoding a therapeutic product into an endogenous pre-mRNA of the non-pluripotent cell induces and repairs the non-pluripotent cell into a repaired pluripotent stem cell.

21. The method of claim 20, wherein said PTM comprises one or more target binding domains that target binding of the PTM to an endogenous pre-mRNA; a 3′ splice region that includes a branch point, pyrimidine tract and a 3′ splice acceptor site and/or 5′ splice donor site; and a spacer region to separate the RNA splice site from the target binding domain.

22. The method of claim 20, wherein said pluripotency factor comprises at least one transcription factor.

23. The method of claim 22, wherein said transcription factor comprises a transcription factor gene product of an OCT family gene, a KLF family gene, a MYC family gene, or a SOX family gene, or a combination thereof.

24. The method of claim 23, wherein said transcription factor comprises at least one of OCT3/4, SOX2, KLF4, c-MYC, NANOG, LIN 28, or any combination thereof.

25. The method of claim 20, wherein the at least one second PTM encodes a therapeutic product enabling the repair or reprogrammation of pre-mRNAs for the correction of diseases such as cystic fibrosis, hemophilia, hypercholesterolemia, alpha1 anti-trypsin defiencies, thalassemia, sickle cell, and diabetes.
